SEQUENCE LISTING (8 sequences)

SEQ ID NO: 1 - DNA, 2187 bp, Homo Sapiens
  CDS (1)..(2187)
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 2 - protein, 728 aa, Homo Sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 3 - DNA, 576 bp, Homo sapiens
  CDS (1)..(576)
atgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtga

SEQ ID NO: 4 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 5 - DNA, 1073 bp, Homo sapiens
  CDS (91)..(558)
ccacgcgtccgaaaaaatactgttggcagcagcacaatgtttgggctaagacctggtcttgaaagcgccacaagcagcagctgctgagccatggctgaaggggaaatcaccaccttcacagccctgaccgagaagtttaatctgcctccagggaattacaagaagcccaaactcctctactgtagcaacgggggccacttcctgaggatccttccggatggcacagtggatgggacaagggacaggagcgaccagcacattcagctgcagctcagtgcggaaagcgtgggggaggtgtatataaagagtaccgagactggccagtacttggccatggacaccgacgggcttttatacggctcacagacaccaaatgaggaatgtttgttcctggaaaggctggaggagaaccattacaacacctatatatccaagaagcatgcagagaagaattggtttgttggcctcaagaagaatgggagctgcaaacgcggtcctcggactcactatggccagaaagcaatcttgtttctccccctgccagtctcttctgattaaagagatctgttctgggtgttgaccactccagagaagtttcgaggggtcctcacctggttgacccaaaaatgttcccttgaccattggctgcgctaacccccagcccacagagcctgaatttgtaagcaacttgcttctaaatgcccagttcacttctttgcagagccttttacccctgcacagtttagaacagagggaccaaattgcttctaggagtcaactggctggccagtctgggtctgggtttggatctccaattgcctcttgcaggctgagtccctcaatgcaaaagtggggctaaatgaagtgtgttaaggggtcagctaagtgggacattagtaactgcacactatttccctctactgagtaaaccctatctgtgattcccccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 6 - protein, 155 aa, Homo sapiens
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 7 - DNA, 7681 bp, Artificial
  pcDNA3.1(-)HGF
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgctctagaactagctggatcctgccagcccgtccagcagcaccatgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgttttccagctggtatatggccctgaaggatcagatctggttttaatgaagcttgccaggcctgctgtcdtggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatagctgttaacccgggtcgaagcggccgccactgtgctggatatctgcagaattccaccacactggactagtggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 8 - DNA, 5181 bp, Artificial
  pVAX1HGF/MGB1
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccagcccgtccagcagcaccatgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatagctgttaacccgggtcgaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

(19) United States
(12) Patent Application Publication
(10) Pub. No.: US 2009/0082263 A1
(43) Pub. Date: Mar. 26, 2009

(54) DRUG AND METHOD FOR IMPROVING BRAIN FUNCTION

(75) Inventors: Yasufumi Kaneda, Osaka (JP); Ryuichi Morishita, Osaka (JP); Munehisa Shimamura, Osaka (JP)

Correspondence Address: ROPES & GRAY LLP, PATENT DOCKETING 39/361, 1211 AVENUE OF THE AMERICAS, NEW YORK, NY, 10036-8704, US

(73) Assignee: AnGes, MG, Inc., Osaka (JP)

(21) Appl. No.: 11/658,897

(22) PCT Filed: Jul. 29, 2005
(86) PCT No.: PCT/JP2005/013949

§ 371 Date: Oct. 29, 2008

(30) Foreign Application Priority Data
Jul. 29, 2004 (JP) 2004-222649

(51) Int. Cl. A61K 38/18; A61K 31/7088; C07K 14/475

(52) U.S. Cl. 514/12; 514/44; 530/350

(57) ABSTRACT

It is intended to provide a novel remedy for improving the brain function or preventing the same from worsening and a novel administration method for the remedy. Namely, a composition for preventing the brain function from worsening or improving the brain function which contains a cell growth factor. It is preferred that this cell growth factor is one selected from the group consisting of vascular endothelial growth factors (VEGFs), fibroblast growth factors (FGFs) and hepatocyte growth factors (HGFs). A method for preventing the brain function from worsening or improving the brain function which comprises the step of administering a cell growth factor to a patient.

### TECHNICAL FIELD

The present invention relates to a medicament and a method for amelioration of a cerebral function. More particularly, the present invention relates to a medicament for amelioration of a cerebral function which contains a cellular growth factor or the like as an active ingredient, and a method for amelioration of a cerebral function.

### BACKGROUND ART

The brain is the most important organ among various organs, but it is also the least studied field. Within this field, control of cerebral functions has not been fully explained in relation to its actual mechanisms. In particular, it is unknown if amelioration of physiological conditions of the brain (for example, neoangiogenesis) will directly result in amelioration of cerebral functions in any ways, and there are a number of unknown points with regard to the control of cerebral functions (for example, amelioration, prevention of deterioration and the like).

Cerebral occlusive diseases, occlusive disease of the circle of Willis and the like caused by atherosclerosis in the artery of the brain often cause chronic reduction in cerebral blood flow. This state may not only cause subsequent cerebral ischemia but also neuropathological alteration including dementia, that is, decline in cerebral functions (Sekhon L H, Morgan M K, Spence I, Weber N C., Stroke 25, 1022-1027 (1994); Stroke 29, 1058-1062 (1998); Stroke 24,259-264 (1993); and Kalaria R N, Bhatti S U, Lust W D, Perry G., Ann. N.Y, Acad. Sci. 695, 190-193 (1993). However, no effective therapy for the amelioration of such decline in cerebral functions has been established.

Development of a new blood vessel or neoangiogenesis is initiated with the activation of endothelial cells in a parent blood vessel. A growth factor which has been shown to stimulate such neoangiogenesis in vivo and possess mitogenic effect on endothelial cells in vitro is referred to as “angiogenic growth factor”. Involvement of angiogenic growth factors in therapy was published for the first time by Folksman et al. in an article (Folksman et al., N. Engl. J. Med. 285, 1182-1186 (1971)). Further, through subsequent studies, it has been confirmed that the development of collateral vessels may be enhanced and/or promoted in animal models with myocardial ischemia or limb ischemia, using a recombinant angiogenic factor, for example, fibroblast growth factor (FGF) family (Science 257, 1401-1403 (1992) and Nature 362, 844-846 (1993)), endothelial cell growth factor (J. Surg. Res, 54, 575-583 (1993)), vascular endothelial growth factor (VEGF) (Circulation 90, II-228-II-234 (1994)) and the like. Furthermore, the present inventors found that HGF also acts as an endothelium-specific growth factor, which is similar to VEGF (J. Hypertens. 14, 1067-1072 (1996)).

Strategy of using an angiogenic growth factor as described above for therapy of vascular disorder is referred to as “therapeutic angiogenesis”. More recently, this strategy has been applied to human ischemic diseases. However, the effectiveness of this strategy for amelioration of the decline in cerebral functions or for the prevention of deterioration thereof is unknown at the present time and is unpredictable from the knowledge obtained so far.

A hepatocyte growth factor (HGF) is a pleiotrophic cytokine which exhibits mitogenic activity, motility enhancing activity and morphogenetic activity on various cells (Nature 342, 440-443 (1989)).

Regarding effects of HGF on brain, the following reports have been made. That is, it is known that HGF and transmembrane tyrosine kinase c-Met/HGF receptor are expressed in various regions in a brain, functional binding between HGF and c-Met enhances survival of neurons in a primarily cultured hippocampus, neurite outgrowth is induced in the development of neurons in vitro (J. Cell. Biol. 126, 485-494 (1994) and Japanese Laid-Open Publication No. 7-89869). Recently, it has been reported that HGF is induced in ischemic neurons (Brain Res. 799, 311-316 (1998)), a recombinant HGF has neuroprotective effects against tardive neuronal death after ischemia in the hippocampus, and that continuous injection of recombinant HGF into a brain is effective for reducing the degree of an infarction (J. Cereb. Blood Flow Metab. 18, 345-348 (1998). In view of these knowledge, it is believed that an HGF can act as an important neurotrophic factor during cerebral ischemia. However, the effectiveness of HGF for the amelioration of cerebral functions is unknown and is unpredictable.

On the other hand, a vascular endothelial growth factor (VEGF) is a dimeric glycoprotein which has a mitogenic effect on an endothelial cell, and also has an ability of enhancing vascular permeability. VEGF has direct and specific mitogenic effects on an endothelial cell (Biochem. Biophys. Res. Commun., 161, 851-858 (1989)). Binding sites of a VEGF including tyrosine kinase receptor, Flt, Flk-1 and KDR are found on endothelial cells, but not on other types of cells, and thus the effects of a VEGF are limited to endothelial cells.

Regarding the effects of VEGF on brain, it has been reported that VEGF is rapidly induced in a brain due to ischemic disorder in the central nervous system (Mol. Cell. Biol. 16, 4604-4613 (1996)), and that administration of a recombinant VEGF to a brain surface was effective for reducing the degree of infarction (J. Cereb. Blood Flow Metab. 18, 887-895 (1998)). However, the exact mechanism has not been clarified, and hence the effectiveness of HGF for the amelioration of cerebral functions is also unknown and unpredictable.

Most effects of FGF on brain are also unknown. Effectiveness of FGF for the amelioration of cerebral functions is unknown and unpredictable.

From another viewpoint, while the above HGF, VEGF, FGF and the like act as cellular growth factors, they are also strong angiogenic growth factors, as described above (J. Cell. Biol. 119, 629-641 (1992)). Accordingly, neoangiogenesis is believed to play an important role in the recovery from cerebral ischemia or in the prevention of future paroxysm. However, it is unknown and unpredictable if neoangiogenesis is related to amelioration of decline in cerebral functions or prevention of deterioration of cerebral functions (for example, amelioration of memory function and amelioration of learning function).

Further, since recombinant angiogenic growth factors are rapidly used up, they must be continuously injected into the brain. This operation is very dangerous and difficult under clinical circumstances. It is thus considered that continuous expression and secretion of angiogenic growth factor in an ischemic brain or therearound using transgenesis would be rational, if possible. However, there has been no cases of application of HGF or VEGF to cerebral ischemic disorders (gene therapy), and almost no study on cerebral functions, which reflects specificity of tissue of a brain, has been performed so far. It is therefore unknown whether HGF and VEGF are effective for the amelioration of cerebral functions in reality.

In the present time, when solutions have been found for almost all diseases, cerebral diseases and disorders may be considered as the last area in which only a few solutions have been found. In particular, the demand for therapy and prophylaxis of cerebral diseases and disorders for the purpose of amelioration of decline in cerebral functions and prevention of deterioration of cerebral functions have increased year after year. Among cerebral diseases, those caused by atherosclerosis in cerebral arteries, occlusive disease of the circle of Willis and the like often cause chronic cerebral hypoperfusion. This not only leads to cerebral ischemic events but also results in neuropathological alterations, including dementia (Sekhon L H, Morgan M K, Spence I, Weber N C., Stroke 25, 1022-1027 (1994): Stroke 29, 1058-1062 (1998): Stroke 24, 259-264 (1993); and Kalaria R N, Bhatti S U, Lust W D, Perry G., Ann. N.Y, Acad. Sci. 695, 190-193 (1993). However, no effective treatment for the amelioration of cerebral functions has been established.

Thus, although it has been reported that an angiogenic growth factor such as vascular endothelial growth factor (VEGF), fibroblast growth factor (FGF) and hepatocyte growth factor (HGF) stimulates the development of a collateral vessel in animal models with cerebral ischemia through preclinical studies (Harrigan M R, Ennis S R, Masada T, Keep R F., Neurosurgery. 2002; 50:589-98; Lyons M K, Anderson R E, Meyer F B., Brain Res. 1991; 558:315-20; and Yoshimura S, et al., Hypertension. 2002; 39:1028-34.), it is unknown and unpredictable if such angiogenic growth f actors are effective for the amelioration of cerebral functions in reality.
Non-Patent Document 1: Sekhon L H, Morgan M K, Spence I, Weber N C., Stroke 25, 1022-1027 (1994)
Non-Patent Document 2: Kurumatani T, Kudo T, Ikura Y, Takeda M., Stroke 29, 1058-1062 (1998)
Non-Patent Document 3: Kudo T, Takeda M, Tanimukai S, Nishimura T., Stroke 24, 259-264 (1993)
Non-Patent Document 4: Kalaria R H, Bhatti S U, Lust W D, Perry G., Ann. N.Y, Acad. Sci. 695, 190-193 (1993)
Non-Patent Document 5: Folksman et al., N. Engl. J. Med. 285, 1182-1186 (1971)
Non-Patent Document 6: Yanagisawa-Miwa A, Uchida Y, et al., Science 257, 1401-1403 (1992)
Non-Patent Document 7: Nabel E G, et al., Nature 362, 844-846 (1993)
Non-Patent Document 8: Pu L Q, Sniderman A D, Arekat Z, Graham A M, Brassard R, Symes J F., J. Surg. Res, 54, 575-583 (1993)
Non-Patent Document 9: Takeshita S, Pu L Q, Stein L A, Sniderman A D, Bunting S, Ferrara N, Isner J M, Symes J F, Circulation 90, II-228-II234 (1994)
Non-Patent Document 10: Y. Nakamura, R. Morishita, J. Higaki, I. Kida, M. Aoki, A. Moriguchi, K. Yamada, S. Hayashi, Y. Yo, H. Nakao, K. Matsumoto, T. Nakamura and T. Ogihhara, J. Hypertens. 14, 1067-1072 (1996)
Non-Patent Document 11: T. Nakamura, T. Nishizawa, M. Hagiya, T. Seki, M. Shimonishi, A. Sugimura, K. Tashiro and S. Shimizu, Nature 342, 440-443 (1989)
Non-Patent Document 12: Jung W, Castren E, Odenthal M, Vande Woude G F, Ishii T, Dicenes H P, Lindholm D, Schirmacher P. J, Cell. Biol. 126, 485-494 (1994)
Non-Patent Document 13: Hayashi T, Abe K, Sakurai M, Itoyama Y., Brain Res. 799, 311-316 (1998)
Non-Patent Document 14: Morita F, Et al., J. Cereb. Blood Flow Metab. 18, 345-348 (1998)
Non-Patent Document 15: Ferrara, N. and Henzel, W J, Biochem. Biophys. Res. Commun., 161, 851-858 (1989)
Non-Patent Document 16: Forsythe, J A, Jiang, B H, Iyer, N V, Agani, F, Leung, S W, Koos, R D, and Semenza, G L, Mol. Cell. Biol., 16, 4604-4613 (1996)
Non-Patent Document 17: Hayashi, T., Abe, K., & Itoyama, Y. J. Cereb. Blood Flow Metab. 18, 887-895 (1998)
Non-Patent Document 18: Bussolino, F., et al., J. Cell. Biol. 119, 629-641 (1992)
Non-Patent Document 19: Harrigan M R, Ennis S R, Masada T, Keep R F., Neurosurgery. 2002; 50:589-98
Non-Patent Document 20: Lyons M K, Anderson R E, Meyer F B., Brain Res. 1991; 558:315-20.
Non-Patent Document 21: Yoshimura S, et al., Hypertension. 2002; 39:1028-34.
Patent Document 1: Japanese Laid-Open Patent Application No. 7-89869

### DISCLOSURE OF THE INVENTION

### Problems to be Solved by the Invention

The problem of the present invention is to provide a novel therapeutic agent for the amelioration of cerebral functions or the prevention of deterioration of cerebral functions and a novel administration method of such a therapeutic agent.

### Means for Solving the Problems

The present invention has solved the above problem by providing a composition containing a growth factor such as HGF (hepatocyte growth factor) gene and/or VEGF (vascular endothelial growth factor) gene as an active ingredient, which has acts as therapy for disorders of cerebral functions or prevention of deterioration of cerebral functions, and enhances neurite outgrowth or synaptogenesis.

Therefore, the present invention relates to the following items:

(1) A composition for prevention of deterioration of a cerebral function or amelioration of a cerebral function, the composition including a cellular growth factor as an active ingredient.
(2) The composition according to item 1, wherein the cellular growth factor has action of inducing vascular growth.
(3) The composition according to item 1, wherein the cellular growth factor is selected from the group consisting of: vascular endothelial growth factor (VEGF); fibroblast growth factor (FGF); and hepatocyte growth factor (HGF).
(4) The composition according to item 1, wherein the cellular growth factor is a hepatocyte growth factor (HGF).
(5) The composition according to item 1, wherein the cerebral function is a cognitive function or a motor function.
(6) The composition according to item 1, wherein the cerebral function is affected by cognitive dysfunction or motor dysfunction due to cerebral infarction, cerebral blood flow disorder, cerebral hemorrhage or cerebrovascular disorder.
(7) The composition according to item 1, wherein the cerebral function is selected from the group consisting of a memory function and a spatial learning function.
(8) The composition according to item 1, wherein prevention of deterioration of the cerebral function or amelioration of the cerebral function is achieved by activation of astrocytes.
(9) The composition according to item 1, wherein the cellular growth factor is provided in the form of a protein or in the form of a nucleic acid.
(10) The composition according to item 1, wherein the cellular growth factor is administered with a viral envelope.
(11) The composition according to item 10, wherein the viral envelope is inactivated.
(12) The composition according to item 10, wherein the viral envelope is an envelope of RNA virus.
(13) The composition according to item 10, wherein the viral envelope is an envelope of Paramyxoviridae virus.
(14) The composition according to item 10, wherein the viral envelope is an envelope of HVJ.
(15) The composition according to item 10, wherein the cellular growth factor is contained in the viral envelope.
(16) The composition according to item 1, which is delivered by administration to subarachnoid space or by intracisternal administration.
(17) The composition according to item 1, which is administered six days after development of cerebral infarction, cerebral blood flow disorder, cerebral hemorrhage, cerebrovascular disorder or disorder of a cerebral function.
(18) The composition according to item 1, which is administered six days after development of cerebral infarction, cerebral blood flow disorder, cerebral hemorrhage, cerebrovascular disorder or disorder of a cerebral function, wherein the cellular growth factor is in the form of a nucleic acid.
(19) A method for prevention of deterioration of a cerebral function or amelioration of a cerebral function, the method including the step of:
(A) administering a cellular growth factor to a patient.
   (20) The method according to item 19, wherein the cellular growth factor has action of inducing vascular growth.
   (21) The method according to item 19, wherein the cellular growth factor is selected from the group consisting of: vascular endothelial growth factor (VEGF); fibroblast growth factor (FGF); and hepatocyte growth factor (HGF).
   (22) The method according to item 19, wherein the cellular growth factor is a hepatocyte growth factor (HGF).
   (23) The method according to item 19, wherein the cellular growth factor is administered with a viral envelope.
   (24) The method according to item 23, wherein the viral envelope is an envelope of HVJ.
   (25) The method according to item 19, wherein the cerebral function is a cognitive function or a motor function.
   (26) The method according to item 19, wherein the cerebral function is affected by cognitive dysfunction or motor dysfunction due to cerebral infarction, cerebral blood flow disorder, cerebral hemorrhage or cerebrovascular disorder.
   (27) The method according to item 19, wherein the cerebral function is selected from the group consisting of a memory function and a spatial learning function.
   (28) The method according to item 19, wherein the cellular growth factor is in the form of a protein or in the form of a nucleic acid.
   (29) The method according to item 19, wherein the cellular growth factor is delivered by administration to subarachnoid space or by intracisternal administration.
   (30) The method according to item 19, wherein the cellular growth factor is administered six days after development of cerebral infarction, cerebral blood flow disorder, cerebral hemorrhage, cerebrovascular disorder or disorder of a cerebral function.
   (31) A use of a cellular growth factor in the production of a medicament for prevention of deterioration of a cerebral function or amelioration of a cerebral function.
   (32) The use according to item 31, wherein the cellular growth factor is selected from the group consisting of: vascular endothelial growth factor (VEGF); fibroblast growth factor (FGF); and hepatocyte growth factor (HGF).
   (33) The use according to item 31, wherein the cellular growth factor is a hepatocyte growth factor (HGF).
   (34) The use according to item 31, wherein the cellular growth factor is in the form of a protein or in the form of a nucleic acid.
   (35) A composition comprising a cellular growth factor used for enhancement of neurite outgrowth or synaptogenesis.
   (36) The composition according to item 35, wherein the cellular growth factor is selected from the group consisting of: vascular endothelial growth factor (VEGF); fibroblast growth factor (FGF); and hepatocyte growth factor (HGF).
   (37) The composition according to item 35, wherein the cellular growth factor is a hepatocyte growth factor (HGF).
   (38) The composition according to item 35, wherein the cellular growth factor is in the form of a protein or in the form of a nucleic acid.
   (39) A method for enhancement of neurite outgrowth or synaptogenesis, the method including the step of:

(A) administering a cellular growth factor to an individual for which enhancement of neurite outgrowth or synaptogenesis is necessary.

(40) The method according to item 39, wherein the cellular growth factor is selected from the group consisting of: vascular endothelial growth factor (VEGF); fibroblast growth factor (FGF); and hepatocyte growth factor (HGF).
(41) The method according to item 39, wherein the cellular growth factor is a hepatocyte growth factor (HGF).
(42) The method according to item 39, wherein the cellular growth factor is in the form of a protein or in the form of a nucleic acid.
(43) A use of a cellular growth factor in the production of a medicament for enhancement of neurite outgrowth or synaptogenesis.
(44) The use according to item 43, wherein the cellular growth factor is selected from the group consisting of: vascular endothelial growth factor (VEGF); fibroblast growth factor (FGF); and hepatocyte growth factor (HGF).
(45) The use according to item 43, wherein the cellular growth factor is a hepatocyte growth factor (HGF).
(46) The use according to item 43, wherein the cellular growth factor is in the form of a protein or in the form of a nucleic acid.

The present inventors performed in vivo studies testing whether cerebral functions can be ameliorated by administration of a cellular growth factor such as HGF, FGF and VEGF. As a result, the present inventors clarified that administration of a cellular growth factor resulted in significant amelioration of cerebral functions such as memory function and spatial learning function, and enhanced neurite outgrowth or synaptogenesis, in particular with regard to the region of cerebral infarction and therearound. The present invention was completed based on the above knowledge.

Hereinafter, preferred embodiments of the present invention will be described. It should be recognized that those skilled in the art can appropriately carry out the embodiments or the like based on the explanation of the present invention and well-known ordinary techniques in the field of the art and can also readily understand actions and effects attained by the present invention.

Therefore, it is understood that these and other advantages of the present invention will be apparent to those skilled in the art by reading and understanding the following detailed description with reference to the attached drawings and the like.

### EFFECTS OF THE INVENTION

The present invention provides a composition for the amelioration of decline in cerebral functions or prevention of deterioration of cerebral functions and enhancement of neurite outgrowth or synaptogenesis, in particular with regard to the region of cerebral infarction or therearound. No such composition which is directly related to the control of cerebral functions or compositions for the enhancement of neurite outgrowth or synaptogenesis, in particular with regard to the region of cerebral infarction or therearound, has been known so far. It thus indicates that the present invention has significant effects which have been conventionally impossible.

### BRIEF DESCRIPTION OF THE DRAWINGS

FIG. 1 shows a protocol of treatment. In order to test therapeutic effects of hepatocyte growth factor (HGF) gene on cognitive dysfunction, HGF was injected seven days after the paroxysm. On day 56 after ischemia, cognitive dysfunction was evaluated by Morris water maze and one-trial passive avoidance learning.

FIG. 2 shows nuclear magnetic resonance image of a brain. (a) shows the degree of infarction of a rat by T2 weighted image. There was no difference between both groups. A region with high intensity indicates the region of infarction. (b) shows representative images of T2 weighted image of the frontal region of rat brain. These images are divided into three types. Type A: a region with high intensity is found in the cortex and basal ganglia. Type B: a region with high intensity is found in the cortex or some portions of the basal ganglia. Type C: a region with high intensity is found in the cortex and some portions of the basal ganglia. (c) shows the number of rats for each type. It indicates that most rats were type A, that is, almost none of the rats were of type B or C. Behavior of type A was analyzed. (d) shows the degree of infarction in type A. No significant difference was found.

FIG. 3 shows sensorimotor deficit and spontaneous activity. (a) shows a score of sensorimotor deficit. Sensorimotor deficit was spontaneously recovered from each group, and there was no significant differences. (b) shows spontaneous activity. For rats subjected to medium cerebral artery occlusion, the number of crossover points increased in the open field, but there was no significant differences between other groups (for vehicle-treated rats, n=15, for HGF-treated rats, n=17 and for sham-treated rats, n=10).

FIG. 4 shows Morris water maze test. (a) shows the invisible platform test. In comparison to the sham-treated rats, almost none of the rats subjected to medium cerebral artery occlusion reached the invisible platform. On day 4-6, it was significant that the HGF-treated rats could reach faster than the vehicle-treated rats. (b) shows the visible platform test. Unlike the invisible platform test, rats from both groups could reach the platform. There was no significant difference between these groups (for vehicle-treated rats, n=15, for HGF-treated rats, n=17, and for sham-treated rats, n=10, *p<0.05).

FIG. 5 shows one-trial passive avoidance learning. (a) shows the acquisition trial. The number of learning sessions required for the acquisition of memory was significantly greater for the rats subjected to medium cerebral artery occlusion. However, no significant differences was observed between vehicle-treated rats and HGF-treated rats. (b) shows the retention trial. Latent period during which the rats stayed in the light chamber was longer for HGF-treated rats than vehicle-treated rats on day 1 and day 3 (for vehicle-treated rats, n=15, for HGF-treated rats, n=17, and for sham-treated rats, n=10, *p<0.05).

FIG. 6 shows the immunohistochemistry of GFAP. It shows representative immunohistochemical images of GFAP in the ipsilateral cortex on day 14 and 56. In a peri-infarcted region of HGF-treated rats (#), immunoreactivity was increased on day 14, and decreased on day 56. IC is the ischemic center (for each group, n=3 and bar=100 μm).

FIG. 7 shows quantitative analysis of MAP-2 positive cell and GFAP-positive cell. It shows quantification of MAP-2 positive cell and GFAP-positive cell in the cortex. While there was no significant difference in the number of MAP2-positive cells between the two groups, immunoreactivity of HGF-treated rats in response to GFAP was higher than vehicle-treated rats on day 14, but lower than vehicle-treated rats on day 56 (PI: peri-infarcted region; C: contralateral region, n=3 for each group. *p<0.05, **p<0.01).

FIG. 8 shows the immunohistochemistry of Cdc42. (a) shows representative immunohistochemical images of Cdc42 in the ipsilateral cortex on day 14. In a peri-infarcted region of HGF-treated rats (#), immunoreactivity with Cdc42 significantly increased (IC: ischemic center, Bar=100 μm). (b) shows the quantitative analysis of Cdc42-immunoreactive cell. The number was counted in the peri-infarcted region (#). For HGF-treated rats, the number of Cdc42-immunoreactive cells increases (n=3 for each group, *p<0.05).

FIG. 9 shows representative images of ALP staining. Coronal sections stained with ALP in the ipsilateral cortex are shown. The structure of arteries in the peri-infarcted region (#) was not changed as of day 14. However, the arteries of HGF-treated rats showed more complicated patterns on day 56 (IC: ischemic center).

FIG. 10 shows quantitative analysis of ALP staining. Quantification of a blood vessel in the cortex stained with ALP is shown. The area and the length of the blood vessels were different on day 14, and those of HGF-treated rats increased significantly on day 56 (n=3, *p<0.05).

FIG. 11 shows representative immunohistochemical images of Cdc42 and synaptophysin in the infarction region and contralateral region when the control gene or HGF gene is administered. (a) shows results of immunostaining the region for Cdc42. The left images of (a) show the infarction region and the right images of (a) show the contralateral region. The upper images of (a) show results in the case of administration of a control gene (vector), and the lower images of (a) show results in the case of administration of a HGF gene. IC is the ischemic center. # shows the peri-infarcted region, and the bar indicates 100 μm. (b) shows the number of Cdc42-positive cells in the infarction region (Pi) and the contralateral region (C). * indicates statistical significance (p<0.05). The cross-hatched portion indicates the HGF gene-administered group and the dotted portion indicates the control vector-administered group. (c) shows the results of immunostaining the region for synaptophysin. The left images of (c) show the infarction region and the right images of (c) show the contralateral region. The upper images of (c) show results of administrating a control gene (vector), and the lower images of (c) show results of administrating a HGF gene. The bar indicates 100 μm. (d) shows the number of synaptophysin-positive cells in the infarction region (Pi) and the contralateral region (C). * indicates statistical significance (p<0.05). The cross-hatched portion indicates the HGF-administered group, and the dotted portion indicates the control vector-administered group.

(Sequence Listing)

SEQ ID NO:1: nucleic acid sequence of HGF
SEQ ID NO:2: amino acid sequence of HGF
SEQ ID NO:3: nucleic acid sequence of VEGF
SEQ ID NO:4: amino acid sequence of VEGF
SEQ ID NO:5 nucleic acid of aFGF
SEQ ID NO:6: amino acid sequence of aFGF
SEQ ID NO:7: pcDNA3.1(−)HGF
SEQ ID NO:8: pVAX1HGF/MGB1

### BEST MODE FOR CARRYING OUT THE INVENTION

Embodiments described below are provided for better understanding of the present invention, and it should be understood that the scope of the present invention should not be limited to the following description. Therefore, it is apparent that those skilled in the art can appropriately make modification within the scope of the present invention considering the description provided herein.

Hereinafter, the present invention will be described with reference to the attached drawings and by way of examples, if necessary. It should be understood that, throughout herein, an expression in a singular form also includes a concept thereof in a plural form, unless particularly mentioned otherwise. Therefore, it should be understood that an expression in a singular form (for example, “a”, “an”, “the” and the like in the English language) includes a concept thereof in a plural form, unless particularly mentioned otherwise. It should also be understood that terms used herein are used in a sense typically used in the field of the art, unless particularly mentioned otherwise. Therefore, unless otherwise defined, all of the technical terms and scientific terms used herein have the same meaning as typically understood by those skilled the art in the field to which the present invention belongs. If the meanings are contradictory, the present specification (including definitions) is prior.

(Terms)

Hereinafter, definitions of the terms particularly used herein will be described.

As used herein, “cerebral function” refers to a complicated activity performed by many parts of a cerebrum. Examples of such a function include language function, cognitive function, motor function, memory function, learning function, attention function and the like.

As used herein, “cognitive function” refers to ability of an organism to recognize identity of other organisms. “Cognitive function” is confirmed by, for example, behavioral tests, spontaneous activity tests, Morris water maze tests and the like.

As used herein, “motor function” refers to management of motion by the nerve or brain. “Motor function” can be confirmed by, for example, behavioral tests, spontaneous activity tests, Morris water maze tests and the like.

As used herein, “memory function” refers to a function of the brain or nervous system to memorize a previously experienced state or information. “Memory function” can be confirmed by, for example, behavioral tests, Morris water maze tests, passive avoidance learning and the like.

As used herein, “learning function” refers to a function of the brain or nervous system to memorize and use a previously experienced state. “Learning function” can be confirmed by, for example, behavioral tests, Morris water maze tests, passive avoidance learning and the like.

“Hepatocyte growth factor” (HGF) used in the present invention is described in, for example, Nature, 342, 440 (1989), Japanese Patent Publication No. 2577091, Biochem. Biophys. Res. Commun., 163, 967 (1989), Biochem. Biophys. Res, Commun., 172, 321 (1990) and the like. Representative sequences will be set forth in SEQ ID NOS:1 and 2 (nucleic acid sequence and amino acid sequence respectively). To express such an HGF, HGF cDNA can be incorporated into an appropriate expression vector, as will be described below (nonviral vector and viral vector). Base sequences of cDNA encoding an HGF here are also registered in a database such as Genbank, in addition to those described in the literatures described above. Accordingly, based on these sequence information, for example, RT-PCR reaction can be performed with respect to a mRNA derived from a liver or leucocyte using an appropriate DNA portion as a PCR primer, thereby cloning a cDNA of an HGF. Those skilled in the art would be able to readily perform such cloning in accordance with a basic reference, for example, Molecular Cloning 2nd Edt., Cold Spring Harbor Laboratory Press (1989) and the like.

Further, an HGF of the present invention is not limited to the above HGF. Any protein may be used as an HGF of the present invention, as long as the expressed protein is a protein having substantially the same action as an HGF. For example, an HGF of the present invention can also encompass a protein having substantially the same action as an HGF and consists of an amino acid sequence including one or a plurality of (preferably, several) amino acid substitution, deletion and/or addition with respect to an amino acid sequence of a well-known HGF protein such as a protein encoded by the above cDNA.

Further, a nucleic acid (DNA and RNA) encoding these proteins can be used instead of the above protein in the present invention. As used herein, “form of protein” with regard to a cellular growth factor refers to a cellular growth factor itself (that is, a protein) or a protein having substantially the same action as the cellular growth factor. “Form of nucleic acid” includes a nucleic acid encoding a cellular growth factor itself or a protein having substantially the same action as the cellular growth factor, or a nucleic acid which hybridizes with such a nucleic acid under stringent conditions (herein, these nucleic acids may be represented as a gene), and indicates a form such that, when administered to a subject, a protein encoded by such a nucleic acid is expressed in the subject. In the case of administration to a subject in a “form of nucleic acid”, a protein encoded by the nucleic acid is expressed in the subject, and the protein attains actions as a cellular growth factor.

“Vascular endothelial growth factor (VEGF)” as used herein is described in, for example, Science, 219, 983 (1983), J. Clin. Invest., 84, 1470 (1989), Biochem. Biophys. Res. Commun., 161, 851 (1989) and the like. Representative sequences will be set forth in SEQ ID NOS:3 and 4 (a nucleic acid sequence and amino acid sequence respectively). A VEGF can be prepared using, for example, a cDNA of VEGF incorporated into an appropriate expression vector, as will be described below (nonviral vector and viral vector). Regarding human VEGF, the existence of four subtypes (VEGF121, VEGF165, VEGF189 and VEGF206) have been reported through alternative splicing in transcription (Science, 219, 983 (1983), J. Clin. Invest., 84, 1470 (1989) and Biochem. Biophys. Res. Commun., 161, 851 (1989)). Although any of these VEGFs can be used in the present invention, VEGF165 gene is preferred in view of its most intense biological activity. Further, similar to the HGF described above, even genes obtained by modification or the like with respect to the genes of these VEGFs are encompassed in the category of VEGF of the present invention, as long as the genes encode a protein having the actions of VEGF.

Those skilled in the art would be able to readily clone VEGF as well as HGF based on the sequence described in a literature (for example, Science, 246, 1306 (1989)) and sequence information registered in databases, and would also be able to readily make modification thereof and the like.

“Fibroblast growth factor (FGF)” as used herein is described in, for example, Diilber M S, et al. (1994) Exp Hematol 22(12):1129-33, and is expressed in various normal cells and cancer cells. As gene products thereof, at least nine associated growth factors, bFGF (basic), aFGF (acidic), FGF3, which is an int-2 gene product, FGF4, which is a K-fgf/hst1 gene product, FGF5, FGF6, which are hst2 gene products, keratinocyte growth factor (KGF), androgen-induced growth factor (AIGF) and FGF9, which have similar structure and functions, such as strong binding to heparin, are known. Representative sequences will be set forth in SEQ ID NOS:5 and 6 (a nucleic acid sequence and amino acid sequence=aFGF respectively).

As used herein, “neoangiogenesis” refers to the formation of a new blood vessel and an activity of such formation.

As used herein, “neoangiogenic action (activity)” refers to the ability of a factor to form a new blood vessel in a target when acting on the target.

As used herein, “neurite outgrowth” refers to the outgrowth of any neurite, and is confirmed by observing if substantial outgrowth of a neurite is present using a microscope or the like.

As used herein, “synapse” refers to the conjugation between neurons or between a neuron and other cells (synaptic connection) or between conjugation sites. Synapse may be observed using an electron microscope. For example, the main features defining a synapse may be (1) the synaptic vesicle, (2) the synaptic cleft, (3) the thickening of presynaptic membrane and subsynaptic membrane (post-synaptic membrane) and the like. Therefore, as used herein, “synaptogenesis” refers to formation of a synapse in a position without synapse. The formation of a synapse can be confirmed by verifying the presence of synapse using the above method.

As used herein, “Hemagglutinating virus of Japan” and “HVJ” refer to virus belonging to Paramyxoviridae or Paramyxovirus having a cellular fusion action and are interchangeably used. M. Kuroya et al. (1953) reported the virus as Hemagglutinating virus of Japan. A genome thereof is a minus-strand RNA consisting of approximately 15500 bases. Viral particle of Hemagglutinating virus of Japan has an envelope, and is polymorphic with a diameter of 150-300 nm. Hemagglutinating virus of Japan has RNA polymerase. This virus is unstable under heat. It aggregates almost every type of erythrocytes and is hemolytic. It proliferates in an embryonated egg and/or cytoplasm of a cultured cell derived from a kidney of various animals. Hemagglutinating virus of Japan tends to cause persistent infection when it infects an established cell. Since it has the ability of fusing various cells, it is broadly used for cellular fusion such as for the formation of heterokaryon and the preparation of a hybrid cell.

As used herein, “(viral) envelope” refers to a membrane structure based on a lipid bilayer surrounding a nucleocapsid present in a specific virus such as Hemagglutinating virus of Japan. An envelope is typically found in viruses which mature by budding from a cell. An envelope generally consists of a small projection structure consisting of spike protein encoded by a viral gene and lipids derived from a host. Therefore, “(viral) envelope vector” is a term used in the case where an envelope is used as a vector (carrier), and it may be interchangeably used herein with (viral) envelope in some cases.

Transfer of a gene (that is, a cellular growth factor of the present invention in the form of nucleic acid) to the central nervous system (CNS) is achieved using various viral vectors including adeno-associated virus (AAV) (Fan D, et al., Neurosci Lett. 1998; 248:61-4), retrovirus, (Franceschini I A, et al., J Neurosci Res. 2001; 65:208-19), adenovirus (Miyaguchi K, Maeda Y, Colin C, Sihag R K., Brain Res Bull. 2000; 51:195-202) and herpes simplex virus (Johnson P A, Yoshida K, Gage F H, Friedmann T., Brain Res Mol Brain Res. 1992; 12:95-102). These vectors have advantages and disadvantages with regard to human gene therapy. Although these methods are effective for gene transfer to the central nervous system in vivo, many problems such as safety and the amount transferred are found in gene therapy, particularly in gene therapy for humans. HVJ-E vector is effective in transfecting a gene to the central nervous system without any apparent toxicity. Hence, HVJ-E vector may be used in the present invention.

In order to solve these problems, the present inventors developed and used HJV (Hemagglutinating Virus of Japan)-envelope vector which is a nonviral vector, as described above. This vector system was developed based on the first-generation HVJ-based gene transfer using a viral envelope and liposome (HVJ liposome method, Yamada K, et al., Am J. Physiol. 1996; 271:R1212-20; Kaneda Y, et al., Exp Cell Res. 1987; 173:56-69). The first-generation HVJ vector has great potential with regard to transfection to the central nervous system of rat and Primate (Yamada K. et al. supra; Hagihara Y, et al., Gene Ther. 2000; 7:759-63) but has theoretical disadvantages such as complicated procedures and difficulty in preservation. HVJ-envelope (HVJ-E) vector, which is a novel nonviral vector system, uses only an envelope of HVJ to transfer exogenous gene.

As used herein, “inactivation” refers to inactivation of a genome, when it is used with regard to virus (for example, Hemagglutinating virus of Japan). Inactivated virus is replication-defective virus. Inactivation is achieved by a method described herein (for example, alkylation and the like). Examples of such a method for inactivation include, but not limited to, a method including the steps of: (a) inactivating virus (for example, HVJ) by treatment with alkylating agent; (b) obtaining concentrate of the virus or inactivated virus; and (c) purifying the virus or inactivated virus by column chromatography and subsequent ultrafiltration, and a method in which the order of these steps are changed.

As used herein, “alkylation” refers to substitution of hydrogen atom of an organic compound with an alkyl group. “Alkylating agent” refers to a compound which provides an alkyl group. Examples of alkylating agent include alkyl halide, dialkyl sulfate, alkyl sulfonate, dialkylzinc. Examples of preferred alkylating agent include, but are not limited to, β-propiolactone, butyrolactone, methyl iodide, ethyl iodide, propyl iodite, methyl bromide, ethyl bromide, propyl bromide, dimethyl sulfate, diethyl sulfate and the like.

According to one aspect, the present invention also relates to a method or therapy for the amelioration of cerebral functions or prevention of deterioration of cerebral functions.

According to another aspect, the present invention relates to a method or therapy for enhancing neurite outgrowth or synaptogenesis (a subject is particularly, but not limited to, those caused in a region of cerebral infarction or therearound).

Administration of a pharmaceutical composition and a medicament of the present invention is achieved orally or parenterally. Examples of parenteral delivery method includes local administration, intraarterial administration (for example, via carotid artery), intramuscular administration, subcutaneous administration, intramedullary administration, administration to a subarachnoid space, intraventricular administration, intravenous administration, intraperitoneal administration, intranasal administration and the like. In the present invention, any route may be used as long as the route delivers the composition to a section to be treated.

As used herein, “gene therapy” or “gene treatment” refers to a method for therapy of diseases by introduction of a nucleic acid (for example, DNA) to a patient. While gene therapy includes a method using the step of injecting a naked nucleic acid, a vector can be used in many cases. In the present invention, a viral envelope is used as a vector. Gene therapy is performed by administration of an expressed or expressible nucleic acid to a subject. In such an embodiment of the present invention, a nucleic acid produces a protein encoded by the nucleic acid, and such a protein mediates therapeutic effects.

Any method for gene therapy used in the field of the art may be used in accordance with the present invention. Exemplary methods are described in common general information manuals such as: Goldspie L et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIBTECH 11(5):155-215 (1993). Generally known DNA recombination techniques are described in: Ausubel et al. (ed.), Current Protocols in Molecular Biology, John Wiley & Sons, NY (1993); and Kriegler, Gene Transfer and Expression, A Laboratory Manual, Stockton Press, NY (1990).

While a viral envelope may be used for the introduction in one embodiment of the present invention, a composition and a medicament of the present invention may contain, in addition to viral envelope, an appropriate, pharmaceutically acceptable carrier including an excipient or other compounds to enhance processing of the envelope, for preparing a pharmaceutically usable preparation.

As used herein, “pharmaceutically acceptable carrier” refers to a substance used in the production of a medicament or veterinary medicine and has no harmful influences on active ingredients. Examples of such a pharmaceutically acceptable carrier include, but not limited to, an emulsifier, a suspending agent, a solvent, an extender, a buffer, a delivery vehicle, a diluent, an excipient and/or a pharmaceutical adjuvant.

A pharmaceutical composition and a medicament of the present invention may be administered in any aseptic and biocompatible pharmaceutical carrier (including, but not limited to, saline, buffered saline solution and water). Any of these molecules may be administered to a patient independently or in combination with other agents, in a pharmaceutical composition mixed with an adjuvant and/or pharmaceutically acceptable carrier. In one embodiment of the present invention, a pharmaceutically acceptable carrier is pharmaceutically inactive.

A pharmaceutical preparation for parenteral administration may be an aqueous solution of an active compound. For injection, a pharmaceutical composition of the present invention may be formulated in an aqueous solution, preferably a physiologically adaptable buffer solution such as Hanks' solution, Ringer's solution or buffered saline solution. Further, a suspended active compound may contain a liposome.

A pharmaceutical composition of the present invention encompasses a composition containing an effective amount of envelope of the present invention for achieving a desired purpose. “Therapeutically effective amount” or “pharmacologically effective amount” is a term sufficiently recognized by those skilled in the art, and refers to an amount of an agent effective for causing a desired pharmacological result. Therefore, a therapeutically effective is an amount sufficient for alleviation of symptoms of a disease to be treated. One useful assay for confirming the effective amount for a predetermined application (for example, therapeutically effective amount) is to measure the degree of recovery from a disease of interest. An actual dose depends on an individual to which a treatment is applied, and is preferably an amount optimized so that a desired effect may be achieved without causing any significant side effects. Determination of a therapeutically effective amount is sufficiently within the ability of those skilled in the art.

For any compound, a therapeutically effective amount may be initially estimated using a cell culture assay or any suitable animal model. Animal models may also be used for determining the desired range of concentration and administration route. Subsequently, a dose and route useful for administration to a human can be determined using such information.

A therapeutically effective amount refers to the amount of envelope to alleviate the degree or state of diseases. Therapeutic effects and toxicity of a compound may be determined based on cell culture or standard pharmaceutical procedures (for example, ED₅₀ (therapeutically effective dose for 50% of a group) and LD₅₀ (lethal dose for 50% of a group). The dose ratio between therapeutic effects and toxic effects is the therapeutic index, and is represented by a ratio of ED₅₀/LD₅₀. Pharmaceutical compositions with a great therapeutic index are preferred. Data obtained from cell culture assays and animal experiments are used formulating a range of amount of composition for use in a human. Such a dose of a compound is preferably within the range of circulating concentration containing ED₅₀ without little or no toxicity. Such a dose varies according to the administration form to be used, susceptibility of a patient and the administration route. For example, dose of an envelope is appropriately selected depending on conditions such as the age of the patient, the type of disease, type of envelope to be used, and the like.

In the case of administration of a cellular growth factor to a human using an envelope vector of the present invention, an envelope vector corresponding to 400-400,000 HAU, preferably 1,200-120,000 HAU, more preferably 4,000-40,000 HAU, may be administered to one subject. An amount of exogenous gene contained in the envelope to be administered may be 2-2,000 μg, preferably 6-600 μg, more preferably 20-200 μg, per subject.

As used herein, “HAU” refers to the activity of virus capable of aggregating 0.5% of chicken erythrocytes. 1 HAU corresponds to almost 24,000,000 viral particles (Okada, Y. et al., Biken Journal 4, 209-213, 1961). The above-mentioned amount may be administered once or several times per day.

An exact amount is selected by individual clinician in view of the patient to be treated. Dose and administration is adjusted so as to provide sufficient level of active parts or desired effects. Another factor which may be considered is the severity of a state of disease (for example, size and position of a tumor; age, body weight, sex of a patient; dietary restriction, frequency, combination of drugs and susceptibility to reaction and resistance/response to therapy in administration). Depending on the half-life and clearance speed of a specific preparation, a pharmaceutical composition with prolonged action may be administered every three or four days, every week, once per two weeks or once per month. Guidance with regard to a specific dose and method of delivery is provided in known literatures in the field of the art.

An amount of a composition and medicament used in a method of the present invention can be readily determined by those skilled in the art by taking into consideration a purpose of use, subject patient (type, severity and the like), age, body weight, sex, case history of the subject, form or type of cytophysiologically active substance, form or type of a cell and the like.

Frequency of application of a method of the present invention to a subject (or a patient) can also be determined readily by those skilled in the art by taking into consideration a purpose of use, a disease to be treated (type, severity and the like), age, body weight, sex, case history and therapeutic progress of the patient. Frequency may be, for example, once per day to once per several months (for example, once per week to once per month). It is preferred to administer once per week to once per month while observing progress.

While a composition and medicament of the present invention may be used for application to a human, they may also be used for other hosts (for example, mammals and the like).

Molecular biological method, biochemical method and microbiological method used herein are well known and commonly used in the field of the art, and are described in, for example, Ausubel F. A. et al. Ed. (1998), Current Protocols in Molecular Biology, Wiley, New York, N.Y.; Sambrook et al. (1987), Molecular Cloning: A Laboratory Manual, 2nd Ed., Cold Spring Harbor Laboratory Press, Cold Spring Harbor, N.Y.; Experimental Medicine, Supplement, “Idenshidonyu & Hatsugen Kaiseki Jikkenho (Experimental Method for Analysis of Gene Introduction & Expression), YODOSHA CO., LTD. (1997) and the like.

According to the present invention, a method for delivering a cellular growth factor to the brain, includes the steps of: 1) temporarily occluding an artery of the head or cervix; and 2) introducing a cellular growth factor to the brain while the artery of the head or cervix is occluded. Here, by temporal occlusion of the artery, a cellular growth factor is effectively (for example, over twice) introduced to the brain. Examples of a method for temporal occlusion include balloon catheter, clipping and the like, and cerebral infarction as a physiological method. Balloon catheter and clipping is preferred. Temporal here means time period sufficient for the administration of a cellular growth factor (for example, at least one min., at least five min. or the like). A preferred time period may be 1-120 min.

The present invention relates to use in the production of a medicament for the treatment of disorder of cerebral functions. In such a use, any embodiments described herein may be used as a preferred embodiment of cellular growth factor.

Although content of DNA in a preparation can be appropriately adjusted depending on the disease to be treated, age and body weight of a patient and the like, it is typically 0.0001-100 mg, preferably 0.001-10 mg, for a DNA of the present invention. Such a preparation is preferably administered once per several days or several months.

### DESCRIPTION OF PREFERRED EMBODIMENTS

Hereinafter, the description of preferred embodiments will be described. It should be understood that these embodiments are for illustration of the present invention, and the scope of the present invention is not limited to such preferred embodiments.

The present invention has revealed for the first time that cellular growth factors such as HGF, VEGF, FGF and the like ameliorates or prevents disorder of cerebral functions. Although results with regard to physiological phenomenon of brain at the level of blood vessel and cells have been conventionally shown, it is revealed for the first time in the present invention that cerebral function (particularly, memory functions and the like) is ameliorated as the entire system. Thus, the present invention has demonstrated amelioration or prevention at the level of function by the introduction of HGF or VEGF. Accordingly, HGF and VEGF are effectively used as an agent for therapy or prevention of various declines or disorders of cerebral functions, such as the decline or disorder of cerebral functions due to cerebral ischemia, reduction in cerebral blood flow and the like.

As a specific example, the present invention is effectively used as an agent for therapy or prevention of the decline or disorder of cerebral functions due to cerebrovascular occlusion, cerebral infarction, cerebral thrombosis, cerebral embolism, cerebral apoplexy (including subarachnoid hemorrhage, transient cerebral ischemia, cerebral arteriosclerosis and the like), cerebral hemorrhage, occlusive disease of the circle of Willis, injury in the head, cerebrovascular dementia, Alzheimer's dementia, sequela of cerebral hemorrhage or cerebral infarction, and the like.

Further, the present invention can also be used as an agent for therapy or prevention of the decline or disorder of cerebral functions due to neurodegenerative diseases such as Alzheimer's disease, senile dementia of Alzheimer type, amyotrophic lateral sclerosis, Parkinson's disease or the like.

According to the present invention, HGF, VEGF, FGF and the like can be respectively used independently or in combination. They can also be used with a gene of other vascular endothelial growth factors. Further, it is also possible to use HGF, VEGF, FGF and the like in the form of nucleic acid and proteins and the like in combination. Preferred combination is a combination of HGF in the form of nucleic acid and HGF protein or VEGF in the form of nucleic acid and VEGF protein. Combination of HGF in the form of nucleic acid and HGF protein is more preferable.

HGF protein used herein may be prepared by any method, as long as the protein has been purified so that it can be used as a medicament, and a commercially available product (for example, TOYOBO CO., LTD., Code No. HGF-101 and the like) may also be used. The cDNA of HGF obtained by the above cloning is inserted into an appropriate expression vector, and the expression vector is introduced into a host cell to obtain a transformant, thereby obtaining a recombinant HGF protein of interest from the culture supernatant of the transformant (see, for example, Nature, 342, 440 (1989), Japanese Patent No. 2577091). VEGF protein can also be obtained in the same manner.

FGF may be those available from Kaken Pharmaceutical Co., Ltd., or may be prepared based on the sequences obtained from GenBank.

Next, specific examples of a method of gene transfer, form of transfer, dose to be transferred used in gene therapy of the present invention will be described.

When a composition containing a growth factor like HGF in the form of nucleic acid, such as an expression vector containing cDNA of HGF in an expressible form is administered to a patient, administration form of such a composition is generally divided into two groups: the case of using a nonviral vector; and the case of using a viral vector. A method for preparing and administering such an expression vector is explained in detail in manuals of experimentation and the like (Experimental Medicine, Supplement, Idenshichiryo no Kisogijutsu (Basic Techniques of Gene Therapy), YODOSHA CO., LTD. (1996), Experimental Medicine, Supplement, Idenshidonyu & Hatsugen Kaiseki Jikkenho (Experimental Method for Analysis of Gene Introduction & Expression), YODOSHA CO., LTD. (1997), Japan Society of Gene Therapy Ed., Idenshichiryo Kaihatsu Kenkyu Handobukku (Handbook for Developments and Researches of Gene Therapy), NTS (1999)). Hereinafter, the methods will be specifically explained.

A. In the case of using a nonviral vector Using a recombinant expression vector obtained by incorporating a gene of interest into a commonly used gene expression vector, the gene of interest can be introduced into a cell or tissue by the following method.

Examples of a method for gene introduction into a cell include lipofection, phosphoric acid-calcium coprecipitation, DEAE-dextran method, direct injection of DNA using a minute glass tube and the like. As a method for Examples of a method for gene introduction into a tissue include gene transfer using internal type liposome, gene introduction using electrostatic type liposome, HVJ-liposome method, improved HVJ-liposome method (HVJ-AVE liposome method), gene introduction mediated by a receptor, transfer of a DNA molecule with a carrier (metal particle) to a cell using a particle gun, direct introduction of naked-DNA, introduction using positively-charged polymer and the like. Applying any of these methods, a recombinant expression vector can be incorporated into a cell.

Among these methods, in the HVJ-liposome method, a DNA is encapsulated in a liposome formed by lipid bilayer, and then the liposome is fused with inactivated Hemagglutinating virus of Japan (HVJ). The HVJ-liposome method is characterized such that fusion activity with a cell membrane is higher than that in a conventional liposome method, and hence, is a preferred form of gene introduction. A method for preparing an HVJ-liposome is described in detail in literatures (Experimental Medicine, Supplement, Idenshichiryo no Kisogijutsu (Basic Techniques of Gene Therapy), YODOSHA CO., LTD. (1996), Experimental Medicine, Supplement, Idenshidonyu & Hatsugen Kaiseki Jikkenho (Experimental Method for Analysis of Gene Introduction & Expression), YODOSHA CO., LTD. (1997), J. Clin. Invest. 93, 1458-1464 (1994), Am. J. Physiol. 271, R1212-1220 (1996)), and is also described in detail in the examples below. Although Z strain (available from ATCC) is preferred as HVJ, basically, other HVJ strains (for example, ATCC VR-907, ATCC VR-105 and the like) can also be used.

Further, direct injection of naked-DNA is the simplest method of all the above methods, and in view of this, it is a preferred introduction method.

As an expression vector used here, any expression vector may be used, as long as it is capable of expressing a gene of interest in vivo. For example, pCAGGS (Gene 108, 193-200 (1991)), pBK-CMV, pcDNA3.1, pZeoSV (Invitrogen and Stratagene) may be used.

B. In the case of using a viral vector As a viral vector, viral vector such as recombinant adenovirus, retrovirus and the like is used in representative methods. More specifically, a gene of interest is introduced into, for example, a DNA virus or RNA virus such as detoxicated retrovirus, adenovirus, adeno-associated virus, herpesvirus, vaccinia virus, poxvirus, poliovirus, Sindbis virus, Hemagglutinating virus of Japan, SV40, immunodeficiency virus (HIV) and the like, and the cell is infected with the virus, thereby introducing the gene into the cell.

Examples of a method for introducing a gene therapy agent into a patient include an in vivo method in which a gene therapy agent is introduced directly into a living body, and an ex vivo method in which a certain type of cell is extracted from a human, and a gene therapy agent is introduced into the cell outside the body, followed by returning the cell into the body (Nikkei Science (Japanese version of Scientific American), April, 1994, pp. 20-45, The Pharmaceuticals Monthly, 36(1), 23-48 (1994), Experimental Medicine, Extra, 12(15) (1994), Japan Society of Gene Therapy Ed., Idenshichiryo Kaihatsu Kenkyu Handobukku (Handbook for Developments and Researches of Gene Therapy), NTS (1999)). In the present invention, the in vivo method is preferred.

As an administration site, an appropriate administration site is selected based on the disease to be treated, symptoms and the like. For example, in addition to the method in which the skull is directly perforated and a gene is introduced therein, administration to the lateral ventricle, administration to the subarachnoid space or intracisternal administration and the like are also included. Among them, administration to the subarachnoid space is an efficient administration method disclosed in the present invention. For the object of the present invention, that is, for the therapy of reducing cerebral blood flow by neoangiogenesis and/or by suppression of cerebral neurocyte death, administration to the subarachnoid space is preferred.

As a form of preparation, various forms of preparation respectively suitable for the above administration form (for example, liquid preparation or the like) may be taken. For example, in the case of an injection containing a gene as an active ingredient, such an injection can be prepared by an ordinary method. For example, it can be prepared by dissolving the active ingredient in an appropriate solvent (buffer solution such as PBS, saline, sterilized water and the like), filtration sterilization using a filter or the like, if necessary, and then filling the solution into an aseptic container. A commonly used carrier or the like may be added to such an injection, if necessary. Further, a liposome such as HVJ-liposome can be in the form of suspensions, frozen formulations, centrifugation-concentrated frozen formulations, and the like.

Further, for availability of a gene around a diseased site, it is also possible to prepare a sustained-release preparation (mini pellet formulations or the like) to be embedded close to the diseased part, or is continuously and gradually administered to the diseased site using an osmotic pump or the like.

Thus, according to one aspect, the present invention provides a composition for the prevention of deterioration of cerebral functions or amelioration of cerebral functions, which contains a cellular growth factor. Although it has been shown that a cellular growth factor supports physical or external amelioration of cerebral vasculature and the like, actual amelioration of functions has not been reported. Further, it is not recognized that functions of the nervous network can be ameliorated by mere external amelioration (for example, neoangiogenesis), and the relationship between the ameliorations is also unclear. Therefore, even if neoangiogenesis caused by a factor is observed, it is not expected that the neoangiogenesis is effective for amelioration of the functions or prevention of deterioration of the functions until amelioration of the functions is actually demonstrated. It is thus recognized that the present invention attains significant effects unexpected by those skilled in the art. The present invention achieves effects of amelioration of cerebral functions or prevention of deterioration of cerebral functions, which could not be achieved conventionally. In the present invention, administration of a cellular growth factor such as HGF to the brain attained amelioration of functions in the Morris water maze test.

According to another aspect, the present invention provides a composition for enhancing neurite outgrowth or synaptogenesis, which contains a cellular growth factor as an active ingredient. Although it has been shown that a cellular growth factor supports physical or external amelioration of the cerebral vasculature and the like, enhancement of neurite outgrowth or synaptogenesis has not been reported. Further, it is not recognized that functions of the nervous network can be ameliorated by mere external amelioration (for example, neoangiogenesis), and the relationship between the ameliorations is also unclear. Therefore, even if neoangiogenesis by a factor is observed, it is not expected for neoangiogenesis to be effective for the amelioration of the functions or prevention of deterioration of the functions until enhancement of neurite outgrowth or synaptogenesis is actually demonstrated. It is thus recognized that the present invention attains significant effects unexpected by those skilled in the art. The present invention achieves effects of enhancement of neurite outgrowth or synaptogenesis, which could not be achieved conventionally. In the present invention, it was found that administration of a cellular growth factor such as HGF to the brain enhanced neurite outgrowth or synaptogenesis.

In one embodiment, a cellular growth factor used in the present invention preferably has action of inducing vascular growth. Examples of such a cellular growth factor representatively include VEGF, FGF, HGF and the like. More preferably, HGF is used.

Such a cellular growth factor may be administered with a viral envelope. Here, while any viral envelope can be used, an inactivated viral envelope is preferably used. While any type of viral envelope may be possible, an envelope of RNA virus may be preferably used. Here, such an viral envelope is preferably an envelope of Paramyxoviridae virus, and more preferably, an envelope of HVJ (hemagglutinating virus of Japan). This is because such an envelope achieves efficient gene introduction and thus is more suitable for gene therapy, although restriction to the theory is not intended. However, it is understood that the present invention is not limited to these envelopes.

In one embodiment, a cellular growth factor used in the present invention is provided in the form contained in a viral envelope.

Cerebral functions of interest in relation to a composition or method of the present invention for which amelioration or prevention of deterioration is intended include cognitive function. Among cerebral functions, representative examples of such a cerebral function include a cerebral function affected by disorder of cognitive function or motor function. Examples of such a disorder include cerebral infarction, disorders caused by cerebral blood flow disorder, cerebral hemorrhage or cerebrovascular disorder which results in cognitive dysfunction or motor dysfunction.

In another embodiment, cerebral functions of interest of the present invention include memory function and spatial learning function. In particular, almost no drug characterized by amelioration of a function selected from the group consisting of memory function and spatial learning function has been known so far, and thus the superiority the present invention should be noted. Although restriction to the theory is not intended, amelioration of such a cerebral function may be achieved by the activation of astrocytes, but not limited thereto.

While a cellular growth factor used in the present invention may be administered in any form, as long as the factor eventually attains the function in the brain, it may be representatively provided in the form of a protein or nucleic acid. When a cellular growth factor is administered in the form of nucleic acid, such a form is referred to as a form of gene therapy.

While a composition of the present invention may be administered through any route as long as the composition is delivered to the brain and achieves amelioration of functions or prevention of deterioration of functions, for example, administration to the subarachnoid space or intracisternal administration is preferred.

One of the features of the present invention is that effects of ameliorating functions were observed even after a long period (for example, over one day, two days, three days, four days, five days, six days, seven days and the like) from the occurrence of disorder of cerebral functions. Thus, the effects of the present invention should be appreciated in that no urgency such as within several hours from the occurrence of disorder is required. In one preferred embodiment, for example, a composition is administered after six days from the development of disorder of cerebral functions (in case where the day of development is counted as day 1, after day 7).

In particular, in one embodiment, the present invention attains effects as a form of gene therapy, even when a cellular growth factor used is in the form of nucleic acid and is administered after six days from the development of disorder of cerebral functions.

According to another aspect, the present invention provides a method for the prevention of deterioration of a cerebral function or amelioration of a cerebral function, which includes the step of: (A) administering a cellular growth factor to a patient. It will be understood that a patient as a subject of the present invention may be any organism as long as the organism (for example, mammals such as Rodentia, Primates and the like) has cerebral functions (higher functions such as memory function or learning function, for example). A subject is preferably primates including human, and is more preferably a human. The present invention has great significance as it proved for the first time that a cellular growth factor is effective for an application for amelioration of cerebral functions or prevention of deterioration of cerebral functions.

Alternatively, according to another aspect, the present invention provides a method for enhancing neurite outgrowth or synaptogenesis, which includes the step of: (A) administering a cellular growth factor to an individual for which enhancement of the neurite outgrowth or synaptogenesis is necessary. It will be understood that a patient as a subject of the present invention may be any organism as long as the organism (for example, mammals such as Rodentia, Primates and the like) has a nervous system (system in which neurite outgrowth or synaptogenesis can be observed). A subject is preferably primates including human, and is more preferably a human. The present invention has great significance as it proved for the first time that a cellular growth factor is effective for an application as an enhancement for neurite outgrowth or synaptogenesis in a cerebral infarction region or therearound.

It will be understood that a cellular growth factor used in this method of the present invention may take a form of any composition as explained herein.

According to another aspect, the present invention provides a use of a cellular growth factor in the production of a medicament for the prevention of deterioration of a cerebral function or amelioration of a cerebral function. It will be understood that a cellular growth factor used in this use of the present invention may take a form of any composition explained herein. As such, a cellular growth factor, a vascular endothelial growth factor (VEGF), a fibroblast growth factor (FGF), a hepatocyte growth factor (HGF) and the like can be preferably used. A hepatocyte growth factor (HGF) can be used most preferably.

Alternatively, according to another aspect, the present invention provides a use of a cellular growth factor in the production of a medicament for the enhancement of neurite outgrowth or synaptogenesis. It will be understood that a cellular growth factor used in this use of the present invention may take a form of any composition explained herein. As such, a cellular growth factor, a vascular endothelial growth factor (VEGF), a fibroblast growth factor (FGF), a hepatocyte growth factor (HGF) and the like can be preferably used. A hepatocyte growth factor (HGF) can be used most preferably.

(Examples of Administration Method)

Next, examples of a method for the actual administration of a gene will be explained. It will be understood that, while the present invention may be implemented as follows, experimentation or administration may be performed in accordance with other embodiments.

### Experiment I

### An Exemplary Experimental Protocol

### Materials and Experimental Methods

### 1) Ligation of Both Sides of the Carotid Artery

For example, male Sprague Dawley rats (350-400 g; Charles River Japan, Atsugi, Japan) were anesthetized with pentobarbital sodium (50 mg/kg, interperitoneally), thereby allowing natural respiration by the rats. Both sides of the carotid artery were exposed by midline incision in the cervix, and could be firmly ligated with a 2-0 silk.

### 2) Preparation of HVJ-Liposome Complex

As a method used for the preparation of HVJ-liposome complex, a technique described in literatures (J. Clin. Invest. 93, 1458-1464 (1994), Am. J. Physiol. 271, R1212-1220 (1996)) can be used. Briefly, phosphatidylserine, phosphatidylcholine and cholesterol can be mixed in a weight ratio of 1:4.8:2. This lipid mixture (10 mg) can be precipitated on a side surface of a flask by removing tetrahydrofuran in a rotary evaporator. Dried lipids can be hydrated in 200 μl of balanced salt solution (BSS; 137 μM NaCl, 5.4 μM KCl and 10 μM Tris-HCl (pH 7.6)) containing an expression vector in which a gene of interest has been inserted. Liposomes for the control group contains an expression vector in which a gene of interest has not been inserted (BSS, 200 μl). Liposomes can be prepared by shaking and ultrasonic treatment of the mixture.

Purified HVJ (Z strain) was inactivated immediately before use thereof by UV irradiation for three minutes (110erg/mm²/sec.). The liposome suspension (0.5 ml, containing 10 mg of lipids) can be mixed with HVJ (10,000 HAU in BSS of a total amount of 4 ml). This mixture was incubated at 4° C. for five min., and was then incubated at 37° C. for 30 min. while gently shaken. Free HVJ was removed from the HVJ-liposome by sucrose density-gradient centrifugation. Top layer of the sucrose gradient can be recovered and used. Final concentration of plasmid DNA was equal to 20 μg/ml as evaluated in accordance with a previous report (J. Clin. Invest. 93, 1458-1464 (1994), Am. J. Physiol. 271, R1212-1220 (1996)). This preparation method is optimized so as to achieve the maximum transfection efficiency.

### 3) Gene Introduction In Vivo

In order to establish an efficient gene introduction method, we can test three different methods for delivering plasmid which forms a complex with the HVJ-liposome, that is: 1) direct injection into the internal carotid artery; 2) injection into the lateral ventricle; and 3) intracisternally injection (to the subarachnoid space).

In the injection into the internal carotid artery, male Sprague Dawley rats (350-400 g) were anesthetized with pentobarbital sodium (50 mg/kg, intraabdominally), and the left common carotid artery were then incised. Thus, a polyethylene catheter (PE-50, ClayAdams, Parsippany, New Jersey) can be introduced (Rakugi and the like). Distal part of the external carotid artery was separated with a provisional ligature for a short time. The HVJ-liposome complex (1 ml) was then injected into the area of the external carotid artery. After injection, the injecting cannula was removed, and then the ligature was loosened, thereby recovering blood flow to the common carotid artery.

In the injection into the lateral ventricle, anesthetized rats can be laid on a stereotaxic frame (Narishige Scientific Instrument Laboratory, Tokyo, Japan), thereby exposing the skull. Specifically designed stainless steel cannula (30 gauge; Becton Dickinson, Franklin Lakes, N.J.) having a Teflon (registered trademark) connector (FEP tube, Bioanalytical Systems, West Lafayette, Indiana) can be introduced into the left lateral ventricle in a manner described in Am. J. Physiol. 271, R1212-1220 (1996). Stereotaxic coordinate can be determined as follows: 1.3 mm behind bregma, 2.1 mm lateral to midline and 3.6 mm under skull surface. The HVJ-liposome complex was injected into the lateral ventricle (20 μl). After injection, the injecting cannula can be removed. It was observed that behavioral change such as convulsion of the extremities or abnormal behavior was not observed in any of the animals subjected to injection.

In injection into a subarachnoid space, each animal was fixed by the head in the decubitus, and the atlantooccipital membranes were exposed by midline incision of the occipital bone. A stainless steel cannula (27 gauge; Becton Dickinson, Franklin Lakes, N.J.) was introduced to the subarachnoid space. The position of the cannula was confirmed. After removing 10 μl of cerebral spinal fluid in order to avoid an increase in the intracerebral pressure, the HVJ-liposome solution (100 μl:100 μg/ml) was carefully injected into the cisterna magna (subarachnoid space) over one minute. Thereafter, the animals were laid with their head down for 30 min. The aseptic procedure can then be completed after administration of antibiotic in a preventive dose (30,000 U of penicillin G).

### 4) Record

For example, using a laser Doppler imager (LDI) for laser Doppler imaging, blood flow can be continuously measured and recorded for two postoperative weeks. In an LDI system (Moore Instruments Ltd., Devon, GB), 2 mW helium-neon laser is incorporated in order to cause a ray to continuously scan the surface of a tissue in a size of 12×12 cm to a depth of 6000 μm. During scanning, hemocytes moving in a vasculature change the vibration frequency of incident light in accordance with the Doppler theory. Since a photodiode concentrates diffuse light in a reverse direction, original variation in light intensity is converted into voltage variation in a range of 0-10V. Output perfusion value of 0 V can be graduated as 0% perfusion, and 10 V can be graduated as 100% perfusion. When scanning is completed and the diffuse light in a reverse direction is concentrated from all of the sites to be measured, an image showing the distribution of blood flow distinguished by different colors is displayed on a television monitor. The perfusion signal is divided into six different sections which are respectively displayed as different colors. A portion with reduction in blood flow or a portion with no perfusion is represented by dark blue, while a portion with the maximum perfusion is represented by red.

Using LDI, perfusion in the brain surface before occlusion, immediately after occlusion, on day 7 and day 14 after occlusion can be recorded. Through a midline incision portion on the scalp, bone window in a size of 12×12 can be formed with an electric drill. Continuous measurement value can be obtained using this bone window. An image distinguished using different colors is recorded, and then analysis is performed by calculating average perfusion value for each rat. In order to take parameters including ambient light and temperature into consideration, the calculated perfusion value can be represented as a ratio of post-treatment (ischemic) brain to pre-treatment (untreated) brain.

### 5) Histopathological Test

After fixation in 3% paraformaldehyde/20% sucrose solution for one day, 25 μm frozen section of the coronal plane can be produced from each 100 μm section for X-gal staining. Staining with X-gal allows the identification of a stained neuron expressing β-galactosidase. For alkaline phosphatase staining (ALP), 25 μm frozen section of coronal plane can be produced from each 100 μm section. These sections are incubated with PBS containing 0.3% hydrogen peroxide to decrease intrinsic peroxidase activity, and then incubated with the first antibody or lectin diluted in PBS containing 10% equine serum at a room temperature for 60 min. After washing in Tris buffered saline solution containing 2% equine serum for three times, biotin-added second antibody suitable for the species and subsequently avidin-biotin peroxidase complex (Vectastain ABC kit, PK6100, Vector Laboratories, Burlingame, Calif.) can be added and the solution can be incubated. Binding of the antibodies can be visualized using diaminobenxidine. It is possible to omit the first antibody and perform staining with an unrelated immunoglobulin which is suitable for the type and class of the antibody for use as a negative control for each antibody.

6) ELISA method for HGF and VEGF in Cerebrospinal Fluid (CSF)

CSF (100 μl) obtained from rats before or 7 or 14 days after occlusion in both sides of the carotid artery were used in these experiments. Rat and human HGF was measured by ELISA kit (Institute of Immunology, Tokyo), and human VEGF can also be measured using ELISA kit (R & D systems, Minneapolis, Minn.).

### 7) Experimental Materials

Human HGF can be obtained for use by cloning human HGF cDNA (Japanese Patent No. 2577091) using an ordinary method and by inserting the clone into an expression vector, pcDNA (available from Invitrogen).

Human VEGF can be obtained for use by cloning human VEGF165 cDNA (Science 246, 1306 (1989)) using an ordinary method and by inserting the clone into an expression vector, pUC-CAGGS.

Human recombinant HGF can be prepared for use by transfecting ovarian cell of a Chinese hamster (ATCC) or C-127 cell (ATCC) with a recombinant expression vector obtained by inserting a human HGF cDNA (Japanese Patent No. 2577091) into an expression vector pcDNA (Invitrogen) and then purifying from the culture medium using an ordinary method.

Administration of the present invention can be performed based on, but not limited to, the above materials and experimental methods.

A patent, patent application, and reference cited herein is incorporated herein as reference of the present specification in its entirety as if the entirety of each literature were specifically described herein.

Although the present invention has been illustrated hereinabove by way of preferred embodiments of the invention, the invention should not be understood as limited to the embodiments. It will be understood that the scope of the present invention is interpreted as only depending on the claims. From the specific description of the preferred embodiments of the present invention, it will be understood that those skilled in the art can carry out the invention within an equivalent scope based on the description and common general knowledge. It will also be understood that a patent, patent application, and reference cited herein should be incorporated herein by reference of the present specification in its entirety as if the entirety per se were specifically described herein.

### EXAMPLES

Hereinafter, the constitution of the present invention will be explained in more detail. The present invention is not limited thereto. In the following examples, commercially available reagents are from TOYOBO CO., LTD. (Osaka, Japan) and the like, unless particularly mentioned otherwise. Mice and the like were obtained from CLEA Japan, Inc. (Kanagawa, Japan) and the like. The animals were treated in accordance with ethical regulations of Osaka University or ethical regulations recommended by the Japanese government.

### Example 1

### Amelioration of Cerebral Functions

### (Method)

### (Preparation of the HVJ-Envelope Vector)

HVJ-envelope vector was prepared in accordance with a known method (Kaneda, Y. et al., Mol Ther 6, 219-26 (2002) and Shimamura, M. et al., Biochem Biophys Res Commun 300, 461-71 (2003)). Briefly, suspended virus (15000 hemagglutination units) was inactivated by UV irradiation (99 mj/cm²), and was mixed with plasmid DNA (400 μg) and 0.3% Triton-X. After centrifugal separation, the resultant was washed in 1 ml of balanced salt solution (BSS; 10 mM Tris-Cl (pH7.5), 137 mM NaCl and 5.4 mM KCl), and thus the surfactant and DNA which were not incorporated were removed. After centrifugal separation, this envelope vector was suspended in 100 μl of phosphate buffered saline (PBS). This vector was kept at 4° C. until use thereof.

(Construction of Plasmid)

In order to prepare HGF expression vector, human HGF cDNA (2.2 kb) was inserted into an eukaryotic expression plasmid using a cytomegalovirus (CMV) promoter/enhancer (Koike, H. et al., Faseb J5, 5 (2003)). Using this promoter/enhancer, reporter genes were expressed in various cell types. The expression can be regarded as constitutive expression. These control vectors were expression vector plasmids with the same structure which contained a promoter but did not contain HGF cDNA. The plasmids were purified using QIAGEN plasmid isolating kit (Qiagen, Hilden, Germany). The prepared sequences is set forth in SEQ ID NO:7 (pcDNA3.1(−)HGF) and SEQ ID NO:8 (pVAX1HGF/MGB1).

(Surgical Procedure)

Wister male rats (270-300 g; Charles River Japan, Atsugi, Japan) were used for this study. In order to produce permanent MCAo models, as described above, poly-L-lysine-coated 4-0 nylon was arranged around the MCA source, thereby occluding the right middle cerebral artery (Nelayev, L. et al., Stroke 27, 1616-22 (1996)). Briefly, the animals were anesthetized with halothane (1-3.5% in a mixture of 70% N₂O and 30% O₂) using a facial mask. Using a feedback-controlled heating pad, the temperature of the rectum and skull was maintained at 37±0.5° C. throughout the surgical procedure (Unique Medical, Tokyo, Japan). Using an operating microscope, the right common carotid artery, right external carotid artery and right internal carotid artery were separated through median incision. 4-0 nylon was inserted from the right external carotid artery, and was inserted therein by 20 mm. The right external carotid artery was then ligated using 6-0 nylon.

Based on a known method, in vivo gene transfer was performed by intercisternal injection (Shimamura, M. et al., 2003, supra). Briefly, the rats were anesthetized with ketamine (Sankyo, Japan) and xylazine (Bayer Ltd., Japan). Each of the animals was fixed by the head with their face down, and the atlantooccipital membrane thereof was exposed through the posterior median incision. A stainless cannula (27 gauge; Becton Dickinson) was then introduced into the cisterna magna (subarchnoid space). After removal of 100 μl of cerebrospinal fluid (CSF), HJV-envelope vector (100 μl) containing human HGF was injected at a speed of 50 μl/min. The animals were then left with their heads down for 30 min.

(Protocol of Treatment and Behavioral Test)

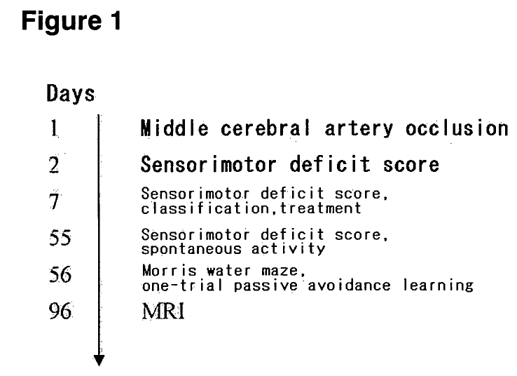

FIG. 1 shows the summary of the protocol of the behavioral test. 10 rats were merely anesthetized (sham operation) and 60 rats were exposed to MCAo (day 1). Based on the neuromuscular functions and body weights evaluated on day 7, these rats were equally divided into vehicle-treated rats (n=23) and HGF-treated rat (n=23). Rats which did not show paralytic symptom on day 7 or those that died by day 7 were excluded from the experiment (n=14). On day 55, alive rats (n=20 for vehicle-treated rats, and n=22 for HGF-treated rats) were evaluated for neuromuscular functions and spontaneous activity. Subsequently, they were tested for cognitive function by the MWM test and passive avoidance learning. On day 96, MRI was performed to evaluate the degree of infarction.

For histopathological analysis on day 14 and day 56, other rats (in the respective experimentations, vehicle-treated rats (n=4) or HGF-treated rats (n=4)) were treated in the same manner as described above, and were killed on day 14 and day 56.

(Sensorimotor Dysfunction)

Among various batteries for sensorimotor dysfunction, the present inventors used a simple protocol for evaluating sensorimotor dysfunction using the following categories (maximum score: 4) (Petullo, D. et al., Life Sci 64, 1099-108 (1999)). Bending of the forelimb: the rats were fixed by the tail on a flat surface. Paralysis of the forelimb was evaluated based on the degree of bending of the left forelimb. Twist of the body: the rats were fixed by the tail on a flat surface. The degree of torsion of the body was studied. Push to a side: the rats were pushed to the left or right side. Rats with right MCA occlusion showed either weakness with respect to the push to the left side or did not show any resistance. Rearrangement of the posterior limb: one of the posterior limbs was removed from the surface. When the limb was removed, rats with right MCA occlusion slowly rearranged or simply did not rearrange the posterior limb at all.

(Spontaneous Activity)

Spontaneous activity was measured through open field test for 30 min. using an automated activity box (Muromachi Kikai, Tokyo, Japan).

(Morris Water Maze Learning)

Water (25° C.) was filled in a cylindrical tank with a diameter of 1.5 m, and a transparent platform with a diameter of 15 cm was laid on a fixed position at the center of one of the four quadrants (O'HARA & CO., LTD., Tokyo, Japan). For adaptation before the test, the rats were made to swim freely for one min. In the invisible platform test, the platform was set under the surface of the water to prevent the rats from seeing the platform. The platform was fixed to one of the quadrants and the starting point was changed for each test. Previous studies show that, when a test is performed twice per day, no difference in the latent period for reaching the platform can be observed between the rats exposed to MCAo 12-14 weeks before and the control rats, until day 6 of the session (Modo, M et al., J Neurosci Methods 104, 99-109 (2000)). Based on these results, the present inventors performed the test twice per day for six days. When a rat could not reach the platform, the latent period was evaluated as 60 sec. In the visible platform test, a flag was put on the platform so as to be seen by the rats. This test was performed twice per day for six days. In this test, the platform and starting point were changed each time. Through these tests, the swimming routes were captured with CCD video camera, and were analyzed by NIH images.

(Passive Avoidance Learning)

In this experimentation, step-through passive avoidance learning was used. An apparatus having a light chamber and a dark chamber (MEDICAL AGENT, Kyoto, Japan) was used. For adaptation, the rats were put into the light chamber, and the door was left open so that the rats could enter the dark chamber. Rats like dark places, and thus have a habit of going toward the dark chamber. In the acquisition trial, the rats were put into the light chamber, and when the rats entered the dark chamber, they were given a shock at 6.0 mA. The respective tests were continued until the rats learn to keep out of the dark chamber for 300 sec. In the memory trial, the rats were placed in the light chamber one, three, five and seven days after the acquisition trial. The present inventors then evaluated the latent period (300 sec. to the maximum) during which the rats stayed in the light chamber.

(Immunohistochemistry)

For immunohistochemistry, the rats were killed and transcardial perfusion fixation was performed in ordinary saline and then in 4% formaldehyde. The brain was extracted, fixed, subjected to cryoprotection treatment, and then sliced into 12 μm or 30 μm sections in a cryostat. After blocking, the section was incubated in 3% goat serum and anti-MAP2 antibody (1:1000, Sigma-Aldrich, Saint Louis, Mo., USA), anti-GFAP antibody (1:1000, Sigma-Aldrich) and anti-Cdc4 antibody 2 (1:500, Santa Cruz, Calif., USA). Thereafter, the section was incubated in an anti-murine fluorescent antibody (1:1000 for NAP2 and GFAP and 1:500 for Cdc42, Alexa Flour 488, Molecular Probes, USA).

For the immunohistochemistry for Cdc42 and synaptophysin in the infarction region and contralateral region, the following antibodies were used: anti-Cdc42 antibody (murine monoclonal antibody; Santa Cruz, Calif., USA); and anti-synaptophysin antibody (murine monoclonal antibody; Chemicon, Temecula, Calif., USA).

(Quantitative Analysis of Immunohistochemistry)

In order to quantify immunoreactivity to GFAP, the obtained image was imported in Adobe Photoshop (version 7.0, Adobe Systems, San Jose, Calif., USA). The color image was converted into a gray scale image. This gray scale image was imported in Mac SCOPE (version 2.5, MITANI CORPORATION, Fukui, Japan). ROI was determined as a region of cerebral cortex adjacent to an infarction region. The number of pixels with a signal equal to or higher than 25 was counted. The immunoreactivity was calculated using the following formula: % region=[number of pixels with a higher signal]/[total number of pixels].

(Alp Staining)

For ALP staining, the section was washed in Tris-HCl and then incubated in a substrate solution (mixture of AS-BI phosphate (Sigma-Aldrich) and fast red violet LB salt (Sigma-Aldrich)) for 30 min.

Five adjacent sections of each rat were observed, and the obtained images were imported in Adobe Photoshop. The color images were converted into gray scale images. Subsequently, ROI was determined as a peri-infarcted region. The area and length of the blood vessel were analyzed using Angiogenesis Image Analyzer (version 1.0, KURABO, Tokyo, Japan).

(Magnetic Resonance Image)

For morphological evaluation of a volume of ischemia, high resolution T2-weighted magnetic resonance image (MRI) (2DFSE, TR=5,000 msec., TE=102 msec.) was obtained using a 3T MRI scanner (Sigma LX VAH/I, GE, Milwaukee, USA). The image was produced with a matrix of 256×256×21 and with a pixel size of 0.39×0.39×1.5 mm.

(Results)

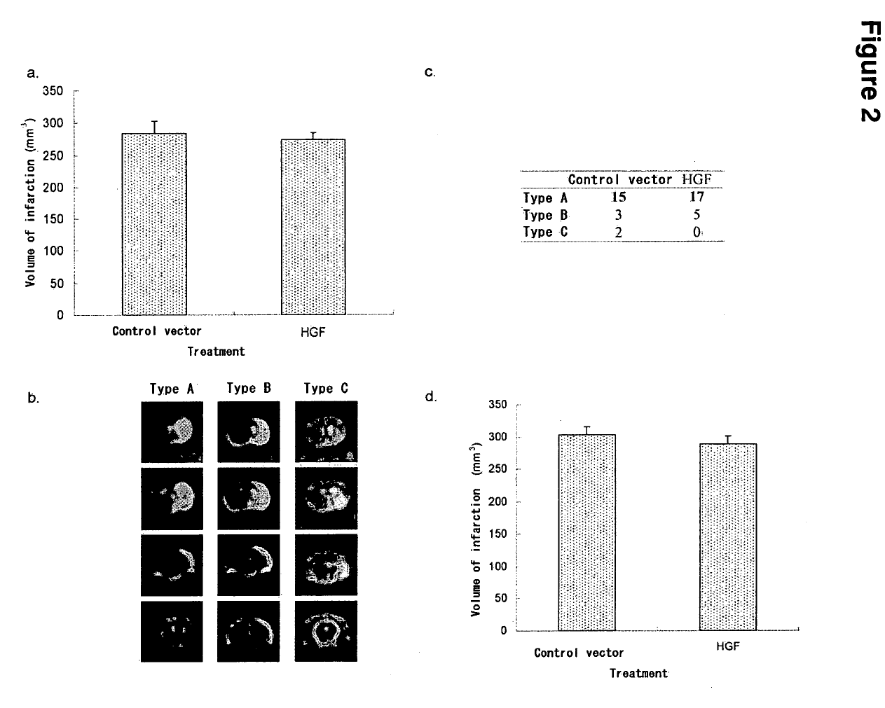

In order to investigate the severity of cerebral infarction, all rats were observed using T2-weighted MRI on day 96 (FIG. 1). Although there was no difference in the total volume of infarction calculated from the T2-weighted image between the groups (FIG. 2a), patters of cerebral infarction were divided into three groups: group in which a region with high intensity is found in the cortex and basal ganglia (type A); group in which a region with high intensity is found in a part of the cortex and the basal ganglia (type B); and a group in which a region with high intensity is found in a smaller region (FIG. 2b and FIG. 2c). In order to eliminate the possibility of diversity of cognitive dysfunction based on the diversity of MCAo models, the present inventors paid attention to the rats of group A in the present study. The present inventors reevaluated the volume of infarction for the rats in group A (FIG. 2d, n=15 (vehicle-treated rats), n=17 (HGF-treated rats)), but there was no significant difference between these groups.

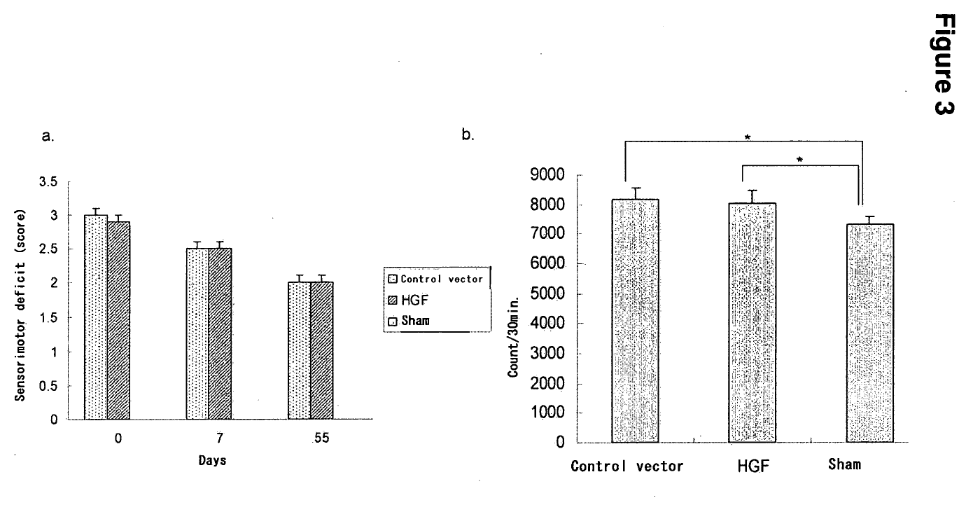

Since sensorimotor dysfunction and spontaneous behavior influences the result of the test for cognitive function to some degree, the present inventors peroformed some tests for them (DeVries, A. C. et al., Neurosci Biobehav. Res. 25, 325-42 (2001)). By day 55, sensorimotor dysfunction was recovered to some degree in both groups, and no difference was observed between the groups (FIG. 3a). In the spontaneous activity test (DeVries, A. C. et al., Supra) for measuring spontaneous activity of the rats, as previously described (Robinson, R. G. et al., Science 205, 707-10 (1979), the rats subjected to MCAo showed increased activity in comparison with the sham-treated rats, but there was no difference between vehicle-treated rats and HGF-treated rats (FIG. 3b).

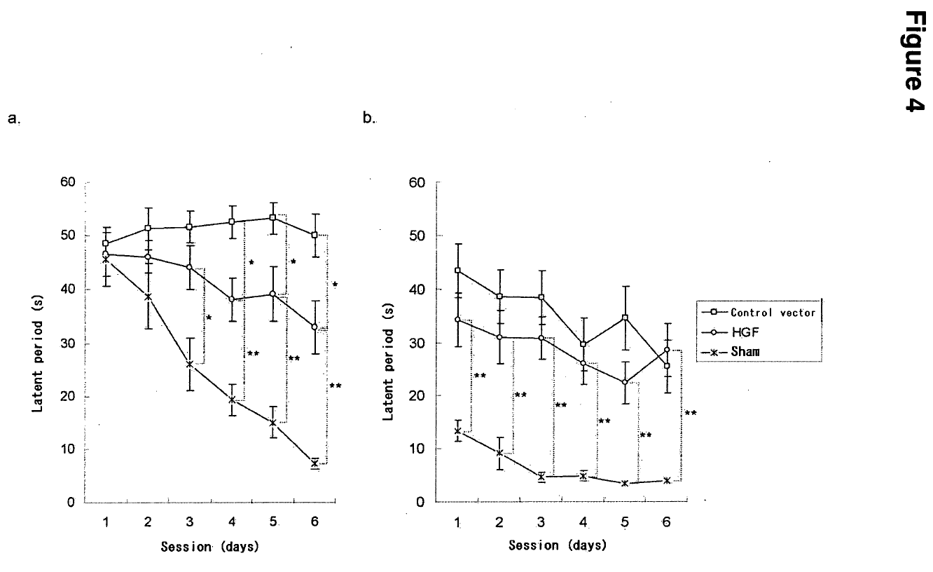

Subsequently, the present inventors performed Morris water maze (MWM) test, thereby evaluating spatial learning and memory (DeVries, A. C. et al., Supra). Although all of the rats subjected to MCAo showed an increase in the latent period before reaching the goal in the MWM test, the latent period was gradually reduced for HGF-treated rats in the invisible platform test (FIG. 4a). In order to eliminate the possibility of influence of loss of vision, sensorimotor dysfunction that will have on the results (DeVries, A. C. et al., Supra), the present inventors also performed a visible platform test. For all of the rats subjected to MCAo, increased latent period was observed, but there was no significant differences between the vehicle-treated rats and HGF-treated rats (FIG. 4b). Throughout the test, no difference in swimming velocity was observed between the vehicle-treated rats and the HGF-treated rats. Accordingly, spatial learning and memory of the HGF-treated rats was partly, but significantly recovered.

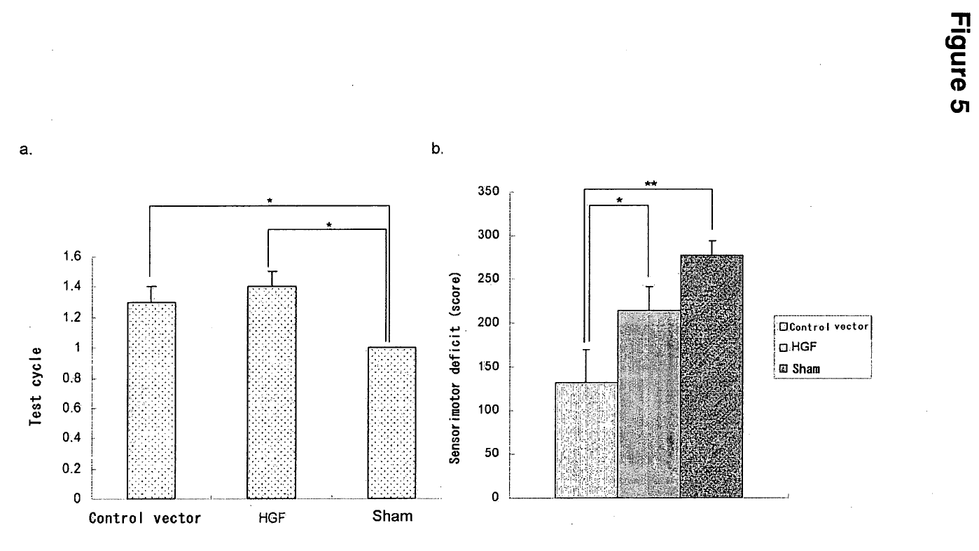

In the passive avoidance learning used for measuring related learning (DeVries, A. C. et al., Supra), the rats subjected to MCAo required more stimulation for acquisition of this learning, and there was no difference between the vehicle-treated rats and HGF-treated rats (FIG. 5a). However, the latent period in the memory trial was significantly longer for the HGF-treated rats, around three days after the acquisition trial. This indicates that the HGF-treated rats may maintain memory longer than the vehicle-treated rats. Thus, it is revealed that the treatment method of the present invention achieves amelioration of cerebral functions.

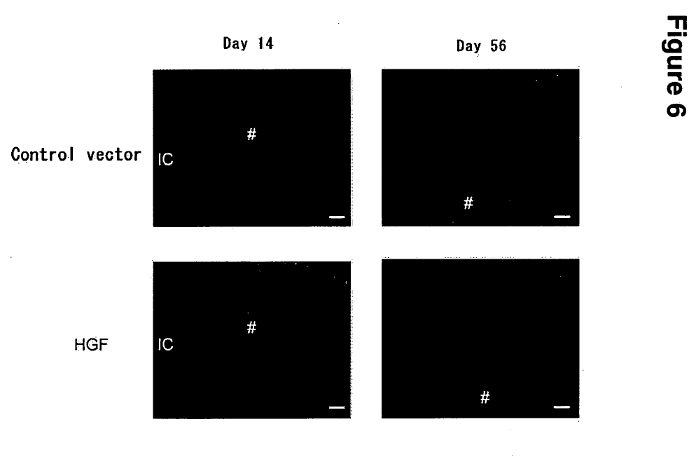

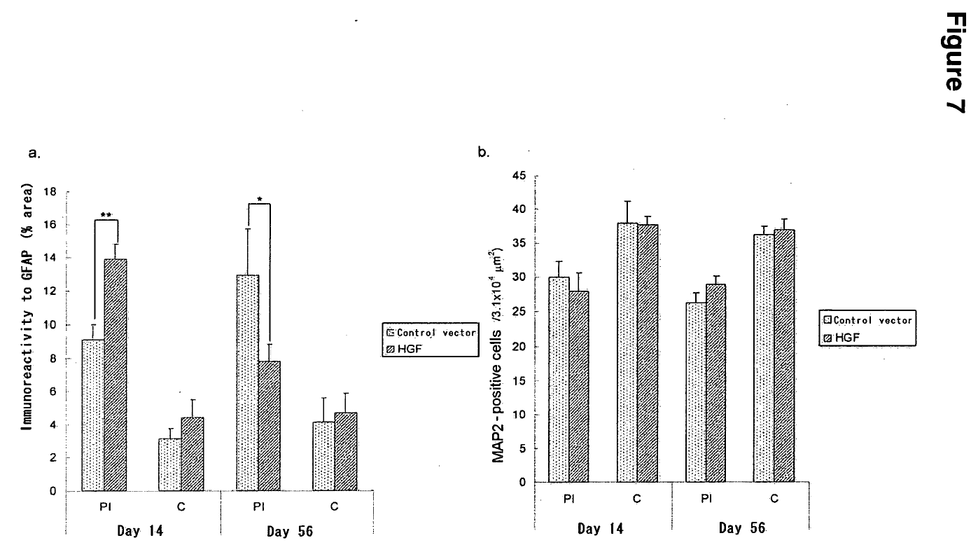

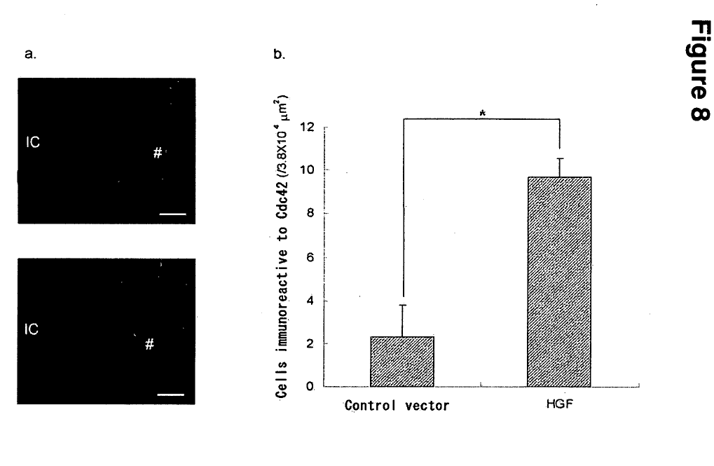

Subsequently, the present inventors attempted to investigate the mechanism of recovery of functions by HGF therapy. For both groups, immunoreactivity to GFAP, a marker of astrocyte, increased in the peri-infarcted region on day 14 and day 56. The immunoreactivity of the HGF-treated rats were higher than the vehicle-treated rats on day 14 and lower on day 56 (FIG. 6 and FIG. 7a). Although cells that are immunoreactive to MAP2, as a marker of matured neuron, decreased in the peri-infarcted region for both groups, there was no significant difference between the groups (FIG. 7b). As previously described, for all of the rats, immunoreactivity to Cdc42 (one of GTPase of Rho family with positive effects on neurite outgrowth) was observed in the hippocampus. Although the peri-infarcted region in the cerebral cortex showed immunoreactivity to Cdc42 for both groups (FIG. 8a), the number of cells immunoreactive to Cdc42 was significantly greater for the HGF-treated rats (FIG. 8b).

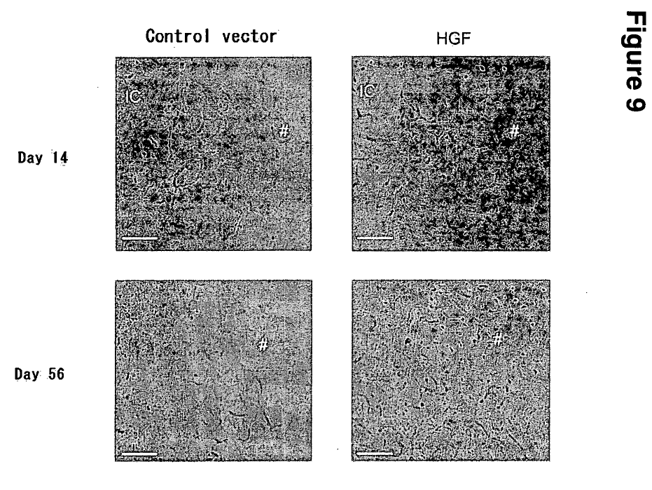

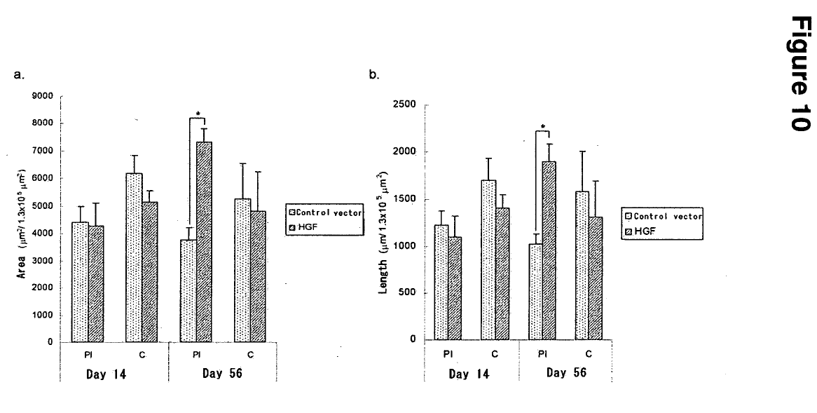

Arteries in the peri-infarcted region and contralateral region on day 14 and day 56 were investigated using ALP staining. In the perieinfarcted region, the number of arteries of the HGF-treated rats on day 56 was significantly increased (FIG. 9). Further, quantitative analysis showed that the arterial region increased and the arteries were elongated on day 56 (FIG. 10). Regarding the contralateral regions, there was no difference between the groups on day 14 and day 56 (FIG. 10).

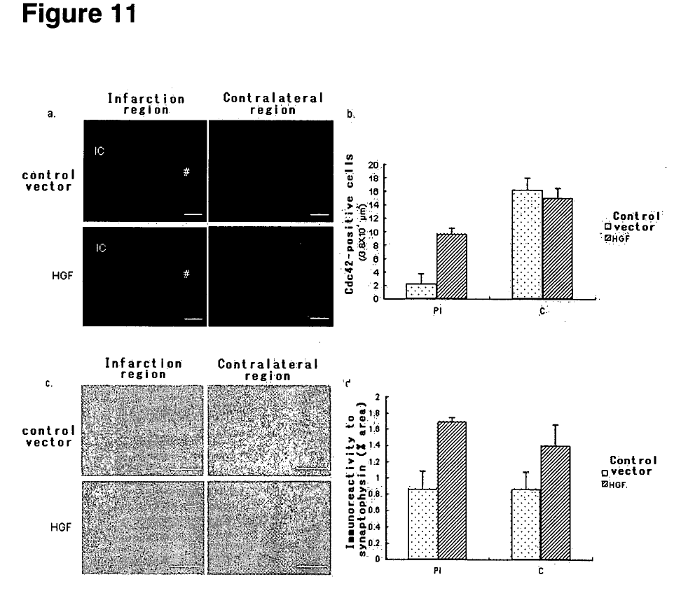

Subsequently, on day 7 of cerebral infarction, a control gene and HGF gene was administered. On day 14 of cerebral infarction, the rats were killed, and immunostaining for Cdc42, which is a small G protein expressed in neurite outgrowth, was performed. On day 56, immunostaining for synaptophysin expressing in a synapse was performed. In both cases, stainability of the HGF-administered group increased in the peri-infarcted regions (FIG. 11).

(Discussion)

The present invention demonstrated that the results from MWM and passive avoidance learning were significantly preferable for the HGF-treated rat on day 56 and that the size and patterns of infarction did not change. Similar to the previous report (Dijkhuzen, R. M. et al., Supra, Zhang, L. et al., J. Neurol. Sci. 174, 141-6 (2000)), sensorimotor dysfunction was spontaneously recovered for the rats subjected to MCAo, and there was no significant difference between the groups. Although some previous studies have showed that neurotrophic factor have useful effects on the recovery from sensorimotor dysfunction within one month after ischemic disorder (Kawamata, T et al., Proc. Natl. Acad. Sci. USA 94, 8179-84 (1997)), the disorder on day 56 in the present studies was not so severe as to show detectable difference. Further, there was no significant differences in the spontaneous activity test and the visible platform test in MWM. These results suggest that the results from MWM and the passive avoidance learning do not reflect the difference in sensorimotor dysfunction or spontaneous activity but difference in learning and memory (DeVries, A. C. et al., Supra). The present inventors consider that the obtained difference depends on functions of the peri-infarcted region in the cerebral cortex. This is because the fact that retention of memory was not observed in spatial learning and passive avoidance in MWM is related to damage to the cerebral cortex (Yonemori, F. et al., J. Cereb Blood Flow Metab. 19, 483-94 (1999), Hirakawa, M. et al., Stroke, 25, 2471-5 (1994)), and there was some histological difference in the peri-infarcted region of the cerebral cortex. Recent studies have shown that the recovery of function is principally related to protection and recovery of hemisphere infarction activity in the cortex (Dikhuiezen, R. M. et al., Supra), and the results from this study are also supported by the recent studies.

Recent reports have revealed that HGF and c-Met are upregulated in astrocytes mainly in the peri-infarcted region after 4-28 days of MCAo, and are related to repair of cerebral tissue after ischemia (Nagayama, T. et al.). From these viewpoints, the present inventors paid attention to the histological alteration in the peri-infarcted region. In the present invention, astrocyte was activated on day 14, but was inactivated on day 56. This is similar to a previous study which has shown effects of BDNF on ischemic disorders (Schabitz, W. R. et al., Stroke 35, 992-7 (2004)). In other words, the astrocyte was temporarily inactivated during the acute stage of the disorder. Initially, activation of astrocytes was believed to be a part of the formation of glial scar which inhibits growth of axon (Eng, L. F. et al., Prog Brain Res 71, 439-55 (1987), Silver, J. et al., Nat. Rev. Neurosci. 5, 146-56 (2004)). However, another report has shown that a temporarily activated astrocyte may enhance neuronal survival and growth of axon (Albrecht, P. J. et al., Expo Neurol 173, 46-62 (2002)). The present inventors consider that exogenous HGF may temporarily activate an astrocyte, enhance the growth of the axon, and/or directly enhance the growth of axon, but such HGF may not cause proliferation of astrocytes to form a scar of astrocyte (Sun, W. et al., Supra). This discussion is supported by increased immunoreactivity to Cdc42, which has positive effects on neurite growth in neuronal cell (O'Kane, E. M. et al., Supra) in the peri-infarcted region. Regrettably, there has not been any report showing in vivo induction of Cdc42 in a neuronal cell by exogenous HGF, but it has been reported that HGF stimulation of endothelial cells leads to activation of Cdc42 with the formation of filopodia and lamellipodia (Royal, I. et al., Mol Biol Cell 11, 1709-25 (2000)). Although further studies are required to clarify the function of HGF in the interaction between astrocytes and axon growth, one possible function is that the reconstruction of neuronal network in the peri-infarcted region may lead to better effects for HGF-treated rats.

Another possible mechanism may be the influence of neoangiogenesis in the peri-infarcted region. Although it is still unknown whether or not the degree of neoangiogenesis is related to functional recovery, a recent report has demonstrated that collateral growth and new capillaries around stroke in the cortex support recovery of perfusion in the ischemic border region and support long-term recovery in rats (Wei, L. et al., Stroke 32, 2179-84 (2001)). Further, it has been reported that some patients who did not experience immediate clinical amelioration by therapy, despite early arterial recanalization, showed delayed clinical amelioration (Alexandrov, A. V. et al., Stroke 35, 449-52 (2004)). The authors of the report suppose recovery of the “stunned brain” by improvement in microenvironment is one of the possible mechanisms. In view of this, recovery of microenvironment in the peri-infarcted region may have some influence on the results, and the increase of arteries in HGF-treated rats may support recovery of cognitive function.

In clinical application to cerebral ischemia, timing for initiating gene therapy is important. Most of the previous reports have shown effects of gene therapy by injecting a gene of interest before the disorder or within several hours after the development of disorder. The purpose of those studies was to inhibit extension of the focus of apotosis and ischemia (Shirakura, M. et al. (2004), Supra, Hayashi, K. et al. (2001), Supra, Yoshimura et al. (2002), Supra, Zhang, W. R. et al. (2002), Supra). Different from studies made so far with regard to gene therapy, the target of the present inventors is to perform reconstruction of neuronal network and neoangiogenesis during the subacute stage to the chronic stage of ischemic disorder. What is important is that the present inventors could achieve recovery of functions by gene therapy at a later timing. In clinical application, it is impossible to administer a gene of interest within several hours, because it takes a long time to prepare a vector containing the gene of interest and to eliminate a malignant tumor prior to the gene therapy using a growth factor. Since “seven days” is a sufficient time period for preparing a vector and evaluating the general state of the tumor, it is recognized that the present studies demonstrated the possibility of gene therapy in clinical application. Further studies are required to clarify the best timing for gene therapy.

The results shown in FIG. 11 shows the possibility of HGF enhancing neurite outgrowth to produce new synaptogenesis. It thus revealed that amelioration of cerebral functions is actually supported by physical alteration of the nerve such as neurite outgrowth and synaptogenesis.

In conclusion, external application of HGF seven days after cerebral ischemia causes more preferable functional effects through the enhancement of growth of axon, neoangiogenesis and inhibition of gliosis in the peri-infarcted region. Although further studies are required for evaluating the safety of a vector and alternative administration route in clinical application, gene therapy using HGF may provide new therapeutic option in the treatment of cerebral ischemia.

### Example 2

### Example of Recovery of Cerebral Function by VEGF

Next, experimentation of recovery of cerebral function by another cellular growth factor was performed. The experimentation was performed in the same manner as described in Example 1 except that VEGF (SEQ ID NO:4) was used instead of the HGF in Example 1. cDNA of human VEGF165 was cloned by an ordinary method (Science 246, 1306 (1989)), and was introduced into an expression vector, pUC-CAGGS.

As a result, amelioration of memory function and spatial learning function is similarly demonstrated.

### Example 3

### Example of Recovery of Cerebral Function by FGF

Next, experimentation of recovery of cerebral function by yet another cellular growth factor was performed. The experimentation was performed in the same manner as described in Example 1 except that aFGF (SEQ ID NO:6) was used instead of the HGF in Example 1. The FGF is available from Kaken Pharmaceutical Co., Ltd.

As a result, amelioration of memory function and spatial learning function is similarly demonstrated.

Although the present invention has been illustrated hereinabove by way of preferred embodiments of the invention, the invention should not be understood as limited to the embodiments. It will be understood that the scope of the present invention is interpreted only depending on claims. From the specific description of preferred embodiments of the present invention, it will be understood that those skilled in the art can carry out the invention within an equivalent scope based on the description and common general knowledge. It will also be understood that a patent, patent application, and reference cited herein should be incorporated herein by reference of the present specification in its entirety as if the entirety per se were specifically described herein.

### INDUSTRIAL APPLICABILITY

The present invention has industrial applicability in the pharmaceutical industry or the like where medicaments for amelioration or prevention of deterioration of cerebral functions are produced.

1. A composition for prevention of deterioration of a cerebral function or amelioration of a cerebral function, the composition comprising a cellular growth factor as an active ingredient.

2. The composition according to claim 1, wherein the cellular growth factor has action of inducing vascular growth.

3. The composition according to claim 1, wherein the cellular growth factor is selected from the group consisting of: vascular endothelial growth factor (VEGF); fibroblast growth factor (FGF); and hepatocyte growth factor (HGF).

4. The composition according to claim 1, wherein the cellular growth factor is a hepatocyte growth factor (HGF).

5. The composition according to claim 1, wherein the cerebral function is a cognitive function or a motor function.

6. The composition according to claim 1, wherein the cerebral function is affected by cognitive dysfunction or motor dysfunction due to cerebral infarction, cerebral blood flow disorder, cerebral hemorrhage or cerebrovascular disorder.

7. The composition according to claim 1, wherein the cerebral function is selected from the group consisting of a memory function and a spatial learning function.

8. The composition according to claim 1, wherein prevention of deterioration of the cerebral function or amelioration of the cerebral function is achieved by activation of astrocytes.

9. The composition according to claim 1, wherein the cellular growth factor is provided in the form of a protein or in the form of a nucleic acid.

10. The composition according to claim 1, wherein the cellular growth factor is administered with a viral envelope.

11. The composition according to claim 10, wherein the viral envelope is inactivated.

12. The composition according to claim 10, wherein the viral envelope is an envelope of RNA virus.

13. The composition according to claim 10, wherein the viral envelope is an envelope of Paramyxoviridae virus.

14. The composition according to claim 10, wherein the viral envelope is an envelope of HVJ.

15. The composition according to claim 10, wherein the cellular growth factor is contained in the viral envelope.

16. The composition according to claim 1, which is delivered by administration to subarachnoid space or by intracisternal administration.

17. The composition according to claim 1, which is administered six days after the development of cerebral infarction, cerebral blood flow disorder, cerebral hemorrhage, cerebrovascular disorder or disorder of a cerebral function.

18. The composition according to claim 1, which is administered six days after the development of cerebral infarction, cerebral blood flow disorder, cerebral hemorrhage, cerebrovascular disorder or disorder of a cerebral function, wherein the cellular growth factor is in the form of a nucleic acid.

19. A method for prevention of deterioration of a cerebral function or amelioration of a cerebral function, the method comprising the step of:
(A) administering a cellular growth factor to a patient.

20. The method according to claim 19, wherein the cellular growth factor has action of inducing vascular growth.

21. The method according to claim 19, wherein the cellular growth factor is selected from the group consisting of: vascular endothelial growth factor (VEGF); fibroblast growth factor (FGF); and hepatocyte growth factor (HGF).

22. The method according to claim 19, wherein the cellular growth factor is a hepatocyte growth factor (HGF).

23. The method according to claim 19, wherein the cellular growth factor is administered with a viral envelope.

24. The method according to claim 23, wherein the viral envelope is an envelope of HVJ.

25. The method according to claim 19, wherein the cerebral function is a cognitive function or a motor function.

26. The method according to claim 19, wherein the cerebral function is affected by cognitive dysfunction or motor dysfunction due to cerebral infarction, cerebral blood flow disorder, cerebral hemorrhage or cerebrovascular disorder.

27. The method according to claim 19, wherein the cerebral function is selected from the group consisting of a memory function and a spatial learning function.

28. The method according to claim 19, wherein the cellular growth factor is in the form of a protein or in the form of a nucleic acid.

29. The method according to claim 19, wherein the cellular growth factor is delivered by administration to subarachnoid space or by intracisternal administration.

30. The method according to claim 19, wherein the cellular growth factor is administered six days after the development of cerebral infarction, cerebral blood flow disorder, cerebral hemorrhage, cerebrovascular disorder or disorder of a cerebral function.

31. A use of a cellular growth factor in the production of a medicament for prevention of deterioration of a cerebral function or amelioration of a cerebral function.

32. The use according to claim 31, wherein the cellular growth factor is selected from the group consisting of: vascular endothelial growth factor (VEGF); fibroblast growth factor (FGF); and hepatocyte growth factor (HGF).

33. The use according to claim 31, wherein the cellular growth factor is a hepatocyte growth factor (HGF).

34. The use according to claim 31, wherein the cellular growth factor is in the form of a protein or in the form of a nucleic acid.

35. A composition comprising a cellular growth factor used for enhancement of neurite outgrowth or synaptogenesis.

36. The composition according to claim 35, wherein the cellular growth factor is selected from the group consisting of: vascular endothelial growth factor (VEGF); fibroblast growth factor (FGF); and hepatocyte growth factor (HGF).

37. The composition according to claim 35, wherein the cellular growth factor is a hepatocyte growth factor (HGF).

38. The composition according to claim 35, wherein the cellular growth factor is in the form of a protein or in the form of a nucleic acid.

39. A method for enhancement of neurite outgrowth or synaptogenesis, the method comprising the step of:
(A) administering a cellular growth factor to an individual for which enhancement of the neurite outgrowth or synaptogenesis is necessary.

40. The method according to claim 39, wherein the cellular growth factor is selected from the group consisting of: vascular endothelial growth factor (VEGF); fibroblast growth factor (FGF); and hepatocyte growth factor (HGF).

41. The method according to claim 39, wherein the cellular growth factor is a hepatocyte growth factor (HGF).

42. The method according to claim 39, wherein the cellular growth factor is in the form of a protein or in the form of a nucleic acid.

43. A use of a cellular growth factor in the production of a medicament for enhancement of neurite outgrowth or synaptogenesis.

44. The use according to claim 43, wherein the cellular growth factor is selected from the group consisting of: vascular endothelial growth factor (VEGF); fibroblast growth factor (FGF); and hepatocyte growth factor (HGF).

45. The use according to claim 43, wherein the cellular growth factor is a hepatocyte growth factor (HGF).

46. The use according to claim 43, wherein the cellular growth factor is in the form of a protein or in the form of a nucleic acid.
